(19) United States
(12) Patent Application Publication
(10) Pub. No.: US 2026/0096798 A1
(43) Pub. Date: Apr. 9, 2026

(54) X-RAY IMAGING APPARATUS

(71) Applicant: SHIMADZU CORPORATION, Kyoto-shi (JP)

(72) Inventor: Takahiro KAWAMURA, Kyoto-shi (JP)

(21) Appl. No.: 19/271,531

(22) Filed: Jul. 16, 2025

(30) Foreign Application Priority Data
Jul. 17, 2024 (JP) 2024-114334
Dec. 25, 2024 (JP) 2024-229097

(51) Int. Cl. A61B 6/00; A61B 6/04; A61B 6/40; A61B 6/46

(52) U.S. Cl. CPC A61B 6/547; A61B 6/0407; A61B 6/4014; A61B 6/463; A61B 6/487

(57) ABSTRACT

An X-ray imaging apparatus 100 comprises an X-ray imaging unit 3 including an X-ray irradiation unit 31 that irradiates X-rays and an X-ray detection unit 32 that detects X rays; a tabletop unit 1 on which a subject 9 is placed and which is movable relative to the X-ray imaging unit 3; a tabletop unit-installed part 2 that is provided on the tabletop unit 1 and includes a material with a lower X-ray transmittance than a portion of the tabletop unit 1 where the subject 9 is placed; a notification unit 6; and a control unit 5 that performs control to cause the notification unit 6 to provide a notification that the tabletop unit-installed part 2 is interfering with an irradiation field, based on irradiation field range information related to a range of an irradiation field of X-rays irradiated from the X-ray irradiation unit 31 and member position information related to a position of the tabletop unit-installed part 2.

### TECHNICAL FIELD

The present invention relates to an X-ray imaging apparatus.

### BACKGROUND ART

Conventionally, X-ray imaging apparatuses are known (see, for example, Patent Literatures 1 and 2).

Patent Literature 1 discloses a patient-side X-ray fluoroscopic imaging apparatus (X-ray imaging apparatus). The X-ray fluoroscopic imaging apparatus disclosed in Patent Literature 1 includes a tabletop (tabletop unit), a first X-ray imaging unit including a first X-ray irradiation unit and a first X-ray detection unit, a second X-ray imaging unit including a second X-ray irradiation unit and a second X-ray detection unit, and an operation unit. X-ray imaging is performed by operating the operation unit. Note that a patient-side X-ray imaging apparatus is an X-ray imaging apparatus that allows a user to perform fluoroscopic imaging and the like at a position close to a subject using an operation unit.

Furthermore, Patent Literature 2 discloses a remote-controlled X-ray imaging apparatus. The X-ray imaging apparatus disclosed in Patent Literature 2 includes an operation unit and a display unit installed in an operation room, and a fluoroscopic imaging table installed in an examination room. The fluoroscopic imaging table includes a tabletop (tabletop unit), an X-ray irradiation unit, and an X-ray detection unit disposed at a position facing the X-ray irradiation unit below the surface of the tabletop. Note that a remote-controlled X-ray imaging apparatus is an X-ray imaging apparatus in which an examination room and an operation room are provided separately, and a user can perform fluoroscopic imaging and the like in the operation room using an operation unit.

### PRIOR ART DOCUMENTS

### Patent Literature

[Patent Literature 1] JP-A-2020-192092
[Patent Literature 2] Japanese Patent No. 7,021,707

### SUMMARY OF THE INVENTION

### Problem to be Solved by the Invention

Although not explicitly stated in Patent Literatures 1 and 2, in the X-ray imaging apparatuses described in Patent Literatures 1 and 2, it is possible to move the tabletop at least in the longitudinal direction of the tabletop, and tabletop moving rails for moving the tabletop in the longitudinal direction are provided at both ends in the transverse direction of the tabletop unit including the tabletop. Furthermore, in both the patient-side X-ray imaging apparatus described in Patent Literature 1 and the remote-controlled X-ray imaging apparatus described in Patent Literature 2, it is possible to move the tabletop unit in its transverse direction. Moreover, in the patient-side X-ray imaging apparatus described in Patent Literature 1, it is possible to move the first detection unit in the transverse direction of the tabletop unit. Here, in both the patient-side X-ray imaging apparatus and the remote-controlled X-ray imaging apparatus, for example, when X-ray imaging of a subject's chest is performed, it may be performed with a long SID (Source to Image Distance). SID refers to the distance from the focal point of the X-ray source to the detector. When the tabletop unit of the patient-side X-ray imaging apparatus or the remote-controlled X-ray imaging apparatus is moved in its transverse direction, or when the first detection unit of the patient-side X-ray imaging apparatus is moved in the transverse direction of the tabletop unit under a situation where the SID is large in X-ray imaging, there is a possibility that the tabletop moving rails of the tabletop unit may appear in the X-ray image. That is, there is a possibility that the tabletop moving rails of the tabletop unit may interfere with the range of the X-ray irradiation field. Due to the interference of the tabletop moving rails (tabletop unit-installed part) with the X-ray irradiation field during X-ray imaging, it may become difficult to see the observation target due to the overlap between the observation target and the tabletop moving rails, or image quality degradation such as insufficient contrast of the observation target may occur in the automatic brightness adjustment processing by the X-ray imaging apparatus. In this case, the radiation exposure dose may increase due to retaking of X-ray images or extension of the fluoroscopic imaging time. Therefore, it is desired to suppress the increase in radiation exposure dose due to retaking of X-ray images or extension of the fluoroscopic imaging time caused by interference of the tabletop unit-installed part with the X-ray irradiation field by making the user aware that the tabletop unit-installed part is interfering with the X-ray irradiation field.

The present invention has been made to solve the above-described problems, and an object of the present invention is to provide an X-ray imaging apparatus capable of suppressing an increase in radiation exposure dose due to retaking of X-ray images or extension of fluoroscopic imaging time caused by interference of a tabletop unit-installed part with an X-ray irradiation field by making a user aware that the tabletop unit-installed part is interfering with the X-ray irradiation field.

### Means for Solving the Problem

An X-ray imaging apparatus according to one aspect of the present invention includes: an X-ray imaging unit including an X-ray irradiation unit that irradiates a subject with X-rays and an X-ray detection unit that detects X-rays transmitted through the subject; a tabletop unit on which the subject is placed and which is movable relative to the X-ray imaging unit; a tabletop unit-installed part that is provided on the tabletop unit and includes a material with a lower X-ray transmittance than a portion of the tabletop unit where the subject is placed; a notification unit; and a control unit that performs control to cause the notification unit to provide a notification that the tabletop unit-installed part is interfering with an irradiation field, based on irradiation field range information related to a range of an irradiation field of X-rays irradiated from the X-ray irradiation unit and member position information related to a position of the tabletop unit-installed part.

### Effects of the Invention

In the X-ray imaging apparatus according to the one aspect, the control unit performs control to cause the notification unit to provide a notification that the tabletop unit-installed part is interfering with the irradiation field, based on the irradiation field range information related to the range of the irradiation field of X-rays irradiated from the X-ray irradiation unit and the member position information related to the position of the tabletop unit-installed part. Thereby, the user can recognize that the tabletop unit-installed part is interfering with the irradiation field because the notification unit provides the notification that the tabletop unit-installed part is interfering with the irradiation field. Therefore, the user can eliminate the interference of the tabletop unit-installed part with the irradiation field. As a result, it is possible to suppress an increase in radiation exposure dose due to retaking of X-ray images or extension of fluoroscopic imaging time caused by interference of the tabletop unit-installed part with the irradiation field.

### BRIEF DESCRIPTION OF THE DRAWINGS

FIG. 1 is a schematic diagram showing the overall configuration of an X-ray imaging apparatus according to a first embodiment.

FIG. 2 is a cross-sectional view taken along line II-II of FIG. 1.

FIG. 3 is a functional block diagram of the X-ray imaging apparatus according to the first embodiment.

FIG. 4 is a schematic diagram of a state before interference in the display of an interference information image related to a tabletop moving rail.

FIG. 5 is a schematic diagram of a state of interference in the display of an interference information image related to the tabletop moving rail.

FIG. 6 is a schematic diagram of a state before interference in the display of an interference information image related to a second X-ray detection unit moving member.

FIG. 7 is a schematic diagram of a state of interference in the display of an interference information image related to the second X-ray detection unit moving member.

FIG. 8 is a functional block diagram of an X-ray imaging apparatus according to a second embodiment.

FIG. 9 is a schematic diagram showing the overall configuration of an X-ray imaging apparatus according to a third embodiment.

FIG. 10 is a functional block diagram of the X-ray imaging apparatus according to the third embodiment.

FIG. 11 is a functional block diagram of an X-ray imaging apparatus according to a fourth embodiment.

FIG. 12 is a schematic diagram of a state before interference in the display of an interference information image related to a tabletop moving rail.

FIG. 13 is a schematic diagram of a state of interference in the display of an interference information image related to the tabletop moving rail.

FIG. 14 is a schematic diagram of a state before interference in the display of an interference information image related to a second X-ray detection unit moving member.

FIG. 15 is a schematic diagram of a state of interference in the display of an interference information image related to the second X-ray detection unit moving member.

FIG. 16 is a schematic diagram showing the overall configuration of an X-ray imaging apparatus according to a first modified example.

FIG. 17 is a functional block diagram of the X-ray imaging apparatus according to the first modified example.

FIG. 18 is a schematic diagram showing a second superimposed image.

FIG. 19 is a schematic diagram showing the overall configuration of an X-ray imaging apparatus according to a second modified example.

FIG. 20 is a schematic diagram showing the overall configuration of an X-ray imaging apparatus according to a third modified example.

### DESCRIPTION OF EMBODIMENTS

Hereinafter, embodiments embodying the present invention will be described with reference to the drawings.

### First Embodiment

### Configuration of X-Ray Imaging Apparatus

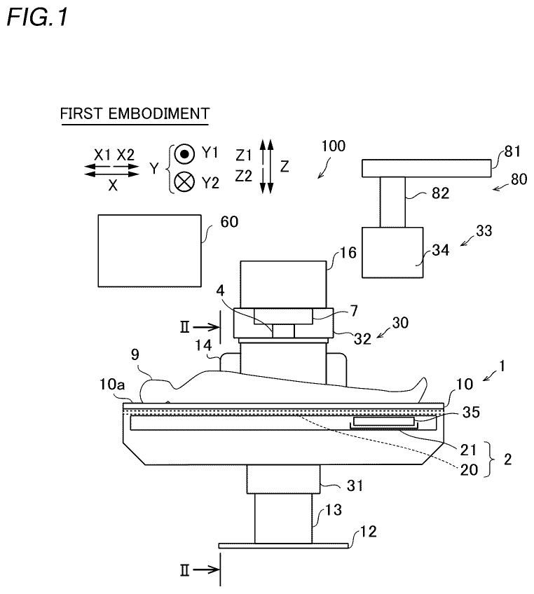

With reference to FIG. 1 to FIG. 7, the configuration of an X-ray imaging apparatus 100 according to the present embodiment will be described.

As shown in FIG. 1, the X-ray imaging apparatus 100 includes a tabletop unit 1, a tabletop unit-installed part 2, a first X-ray imaging unit 30, a second X-ray imaging unit 33, an optical imaging unit 4, a control unit 5 (see FIG. 3), a notification unit 6 (see FIG. 3), and an operation unit 7. The X-ray imaging apparatus 100 is configured to irradiate a subject 9 with X-rays and acquire an X-ray image.

The tabletop unit 1 includes a tabletop 10 and a tabletop support unit 11. The tabletop 10 is configured to have the subject 9 placed thereon. The tabletop 10 has a placement surface 10a on which the subject 9 is placed. In this specification, the X direction is the longitudinal direction of the tabletop 10 when the tabletop 10 is in a horizontal state. The Y direction is the transverse direction of the tabletop 10 when the tabletop 10 is in a horizontal state. The Z direction is the vertical direction.

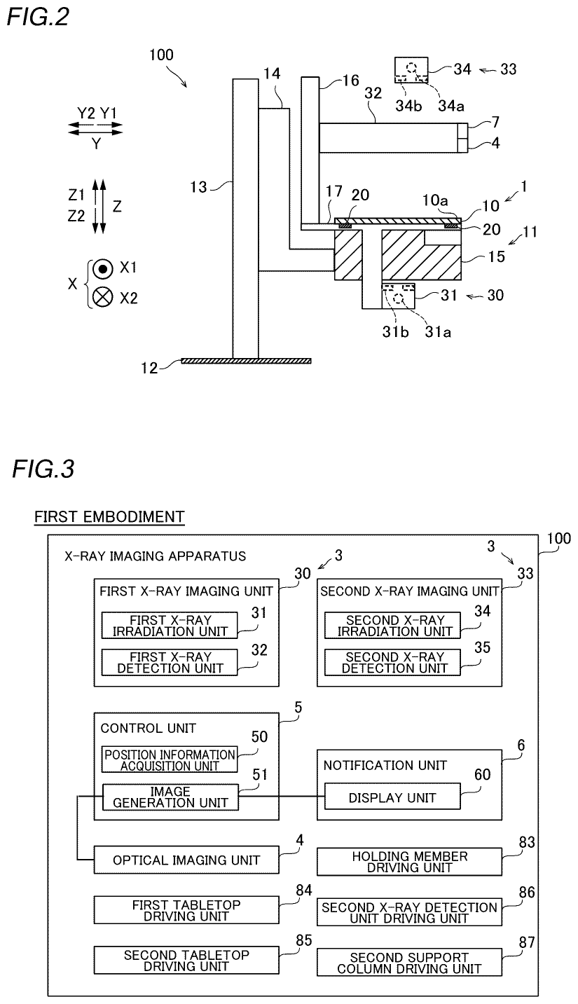

FIG. 2 is a cross-sectional view taken along line II-II of FIG. 1. As shown in FIG. 2, the tabletop support unit 11 includes a base 12, a first support column 13, a holding member 14, a main frame 15, and a second support column 16. In FIG. 2, for convenience of explanation, the illustration of the subject 9 and the illustration of the second X-ray imaging unit 33 except for the second X-ray irradiation unit 34 are omitted. In the X-ray imaging apparatus 100, the first support column 13 is fixed to the base 12, and a main frame 15, on which the tabletop 10 is mounted, is held by the first support column 13 via the holding member 14. A second support column 16 is further held on the main frame 15 via a connection member 17, and the main frame 15, the second support column 16, the holding member 14, and the first support column 13 are arranged in this order from the Y1 side toward the Y2 direction. The tabletop 10 is supported by the main frame 15 via a tabletop moving rail 20, which will be described later. Note that FIG. 2 is an example of the structure of the X-ray imaging apparatus 100.

The tabletop 10 is configured to move up and down (Z direction) together with the second support column 16 and the main frame 15 as the holding member 14 moves up and down (Z direction) around the first support column 13 as an axis by a holding member drive unit 83. The tabletop 10 is also configured to rotate together with the second support column 16 and the main frame 15 as the holding member 14 rotates around the first support column 13 as an axis by a rotation mechanism (not shown).

The tabletop 10 is configured to slide and move in the longitudinal direction (X direction) of the tabletop unit 1 by a first tabletop drive unit 84 via the tabletop moving rail 20. The tabletop 10 is also configured to slide and move in the transverse direction (Y direction) of the tabletop unit 1 by a second tabletop drive unit 85 via a moving member (not shown). That is, the tabletop 10 is configured to be movable relative to the first X-ray imaging unit 30 and the second X-ray imaging unit 33.

As shown in FIG. 1, the tabletop unit-installed part 2 is provided on the tabletop unit 1. The tabletop unit-installed part 2 is disposed between the first X-ray irradiation unit 31 and the first X-ray detection unit 32. The tabletop unit-installed part 2 includes a tabletop moving rail 20 and a second X-ray detection unit moving member 21. The tabletop unit-installed part 2 is configured to include a material with a lower X-ray transmittance than a portion of the tabletop unit 1 where the subject 9 is placed. Specifically, the tabletop unit-installed part 2 is configured to include a metal portion with a lower X-ray transmittance than the portion of the tabletop unit 1 where the subject 9 is placed.

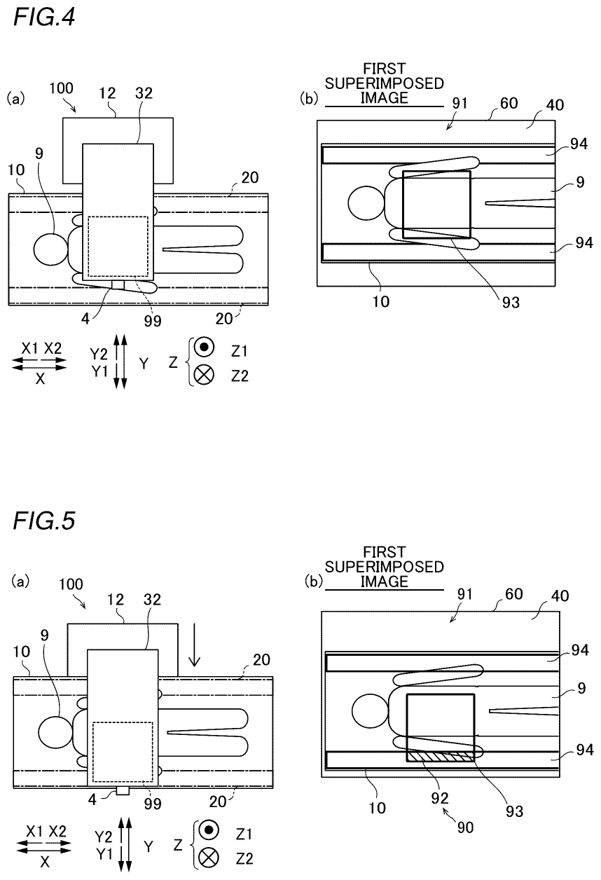

The tabletop moving rail 20 (see FIG. 4(a)) is provided at both ends in the transverse direction of the tabletop unit 1. The tabletop moving rail 20 is provided below the tabletop 10. The tabletop moving rail 20 is configured to engage with an engaging part provided on the surface of the tabletop 10 opposite to the placement surface 10a, and to slide and move the tabletop 10 in the longitudinal direction (X direction) of the tabletop unit 1. The tabletop moving rail 20 is formed of a metal material.

The second X-ray detection unit moving member 21 is provided on the tabletop unit 1. The second X-ray detection unit moving member 21 is provided below the tabletop 10. The second X-ray detection unit moving member 21 holds a second X-ray detection unit 35 placed on the tabletop unit 1. The second X-ray detection unit moving member 21 is configured to move in the longitudinal direction (X direction) of the tabletop unit 1 below the tabletop 10, together with the held second X-ray detection unit 35. The second X-ray detection unit moving member 21 is moved in the longitudinal direction (X direction) of the tabletop unit 1 by a second X-ray detection unit drive unit 86. The second X-ray detection unit moving member 21 is, for example, a metal tray. The second X-ray detection unit moving member 21 is included in an X-ray detection unit moving member.

The first X-ray imaging unit 30 and the second X-ray imaging unit 33 are used to perform fluoroscopic imaging or X-ray imaging of an imaging region of the subject 9 placed on the tabletop 10. Here, fluoroscopic imaging refers to a method of acquiring a moving image of an imaging region while irradiating it with X-rays. X-ray imaging refers to a method of acquiring a still image of an imaging region by irradiating it with X-rays.

The first X-ray imaging unit 30 includes a first X-ray irradiation unit 31 and a first X-ray detection unit 32. The second X-ray imaging unit 33 includes a second X-ray irradiation unit 34 and a second X-ray detection unit 35. The first X-ray imaging unit 30 and the second X-ray imaging unit 33 are included in an X-ray imaging unit 3. The first X-ray imaging unit 30 and the second X-ray imaging unit 33 are an example of the “X-ray imaging unit” in the claims. The first X-ray irradiation unit 31 and the second X-ray irradiation unit 34 are an example of the “X-ray irradiation unit” in the claims. The first X-ray detection unit 32 and the second X-ray detection unit 35 are an example of the “X-ray detection unit” in the claims.

The first X-ray imaging unit 30 includes a first X-ray irradiation unit 31 disposed on the tabletop unit 1, and a first X-ray detection unit 32 disposed at a position facing the first X-ray irradiation unit 31 across the tabletop 10. The first X-ray imaging unit 30 is held by the second support column 16. The first X-ray irradiation unit 31 is disposed on the Z2 side of the second support column 16, and the first X-ray detection unit 32 is disposed on the Z1 side via the connection member 17 (see FIG. 2).

A second support column drive unit 87 is provided on the second support column 16. The second support column drive unit 87 includes a motor and is configured to cause the second support column 16 to move linearly in the longitudinal direction (X direction) of the tabletop 10 and the transverse direction (Y direction) of the tabletop 10. Thereby, the first X-ray imaging unit 30, including the first X-ray irradiation unit 31 and the first X-ray detection unit 32 supported by the second support column 16, is configured to move linearly and integrally while maintaining a facing state. That is, the first X-ray imaging unit 30 is configured to be movable relative to the tabletop 10.

The first X-ray irradiation unit 31 is provided on the side (Z2 side) opposite to the placement surface 10a of the tabletop 10. The first X-ray irradiation unit 31 is held by the second support column 16. As the second support column 16 is moved in the longitudinal direction (X direction) and the transverse direction (Y direction) of the tabletop 10, the position of the first X-ray irradiation unit 31 in the longitudinal direction (X direction) and the transverse direction (Y direction) of the tabletop 10 changes.

The first X-ray irradiation unit 31 includes an X-ray tube 31a (see FIG. 2) and a collimator 31b (see FIG. 2). The X-ray tube 31a irradiates X-rays toward the first X-ray detection unit 32. The X-ray tube 31a irradiates X-rays in the Z1 direction. The collimator 31b includes a plurality of shielding members. The collimator 31b is configured to adjust a first irradiation field of X-rays irradiated from the X-ray tube 31a included in the first X-ray irradiation unit 31 by means of the plurality of shielding members. In this specification, the X-ray irradiation field means a space where X-rays are irradiated between the X-ray tube of the X-ray irradiation unit and the X-ray detection unit. Therefore, the first irradiation field is a space where X-rays are irradiated between the X-ray tube 31a of the first X-ray irradiation unit 31 and the first X-ray detection unit 32.

The first X-ray detection unit 32 is provided at a position (Z1 side) facing the first X-ray irradiation unit 31 across the tabletop 10. The first X-ray detection unit 32 is, for example, an FPD (Flat Panel Detector). The first X-ray detection unit 32 is configured to detect X-rays irradiated from the first X-ray irradiation unit 31 and transmitted through the subject 9. The first X-ray detection unit 32 is not limited to a flat panel detector and may be an image intensifier.

The first X-ray detection unit 32 is held by the second support column 16 via the connection member 17 (see FIG. 2). As the second support column 16 is moved in the longitudinal direction (X direction) and the transverse direction (Y direction) of the tabletop 10, the position of the first X-ray detection unit 32 in the longitudinal direction (X direction) and the transverse direction (Y direction) of the tabletop 10 changes. The first X-ray detection unit 32 is attached with a handle that a user can grip, and via the handle, the user moves the second support column 16 in the longitudinal direction (X direction) and the transverse direction (Y direction) of the tabletop 10. Since the second support column drive unit 87 that drives the second support column 16 has a motor, the user can easily move the first X-ray imaging unit 30 in the longitudinal direction (X direction) and the transverse direction (Y direction) of the tabletop 10.

The second X-ray imaging unit 33 includes a second X-ray detection unit 35 disposed on the tabletop unit 1, and a second X-ray irradiation unit 34 disposed on the side opposite to the second X-ray detection unit 35 across the tabletop 10.

The second X-ray detection unit 35 is disposed on the side (Z2 side) opposite to the placement surface 10a of the tabletop 10. The second X-ray detection unit 35 is located between the first X-ray irradiation unit 31 and the tabletop 10 in the Z direction. The second X-ray detection unit 35 is placed on the second X-ray detection unit moving member 21. The second X-ray detection unit 35 is, for example, an FPD (Flat Panel Detector). The second X-ray detection unit 35 is configured to detect X-rays irradiated from the second X-ray irradiation unit 34 and transmitted through the subject 9.

The second X-ray irradiation unit 34 is supported by a second X-ray irradiation unit moving mechanism 80 provided on the ceiling of the room where the X-ray imaging apparatus 100 is placed. The second X-ray irradiation unit moving mechanism 80 includes a rail 81 and an extendable support part 82. The second X-ray irradiation unit 34 is configured to be movable in the horizontal directions (X direction and Y direction) by the rail 81 of the second X-ray irradiation unit moving mechanism 80, and is configured to be movable in the vertical direction (Z direction) by the support part 82 of the second X-ray irradiation unit moving mechanism 80. The second X-ray irradiation unit 34 is attached with a handle that a user can grip, and via the handle, the user can move the second X-ray irradiation unit 34 in the X, Y, and Z directions. That is, the second X-ray imaging unit 33 is configured to be movable relative to the tabletop 10. Since the second X-ray irradiation unit drive unit 88 that moves the second X-ray irradiation unit 34 has a motor, the user can easily move the second X-ray irradiation unit 34. The user can also adjust the irradiation angle of the second X-ray irradiation unit 34 with respect to the second X-ray detection unit 35 via the handle.

The second X-ray irradiation unit 34 includes an X-ray tube 34a, a collimator 34b, and an irradiation field lamp (not shown). The X-ray tube 34a irradiates X-rays toward the second X-ray detection unit 35. The X-ray tube 34a irradiates X-rays in the Z2 direction. The collimator 34b includes a plurality of shielding members. The collimator 34b is configured to adjust a second irradiation field of X-rays irradiated from the X-ray tube 34a included in the second X-ray irradiation unit 34 by means of the plurality of shielding members. The second irradiation field is a space where X-rays are irradiated between the X-ray tube 34a of the second X-ray irradiation unit 34 and the second X-ray detection unit 35. The irradiation field lamp is provided on the collimator 34b. The irradiation field lamp includes a visible light source. The second irradiation field can be confirmed without using X-rays by the visible light irradiated from the irradiation field lamp.

The optical imaging unit 4 is configured to capture an optical image 40. The optical imaging unit 4 captures an optical image 40 of the subject 9 placed on the tabletop 10 and the tabletop unit 1. In this specification, “optical image 40” means an image obtained by optically detecting visible light reflected by the subject 9 and the tabletop unit 1. The optical imaging unit 4 includes an optical camera. The optical imaging unit 4 is mounted on the first X-ray detection unit 32. The optical imaging unit 4 is mounted facing the direction of the first X-ray irradiation unit 31. The optical imaging unit 4 can capture an optical image 40 of the subject 9 and the direction of the first X-ray irradiation unit 31 from the first X-ray detection unit 32 side.

The optical imaging unit 4 images the subject 9 at least during fluoroscopic imaging by the first X-ray imaging unit 30. The optical imaging unit 4 may also image the subject 9 before fluoroscopic imaging by the first X-ray imaging unit 30. The optical imaging unit 4 may also image the subject 9 after X-ray imaging by the first X-ray imaging unit 30. The imaging range of the optical imaging unit 4 is set to include the range of the X-ray irradiation field and to be larger than the range of the X-ray irradiation field.

As shown in FIG. 3, the notification unit 6 is configured to provide a notification under the control of the control unit 5. The notification unit 6 includes a display unit 60. The display unit 60 is, for example, a liquid crystal display device. The display unit 60 displays interference information 90 under the control of the control unit 5. Specifically, the display unit 60 displays an interference information image 92 as the interference information 90 superimposed on an optical image 40 captured by the optical imaging unit 4, under the control of the control unit 5. The interference information 90 and the interference information image 92 will be described later.

The control unit 5 is configured to acquire irradiation field range information related to the range of the irradiation field of X-rays irradiated from the X-ray irradiation unit and member position information related to the position of the tabletop unit-installed part 2. The control unit 5 is also configured to perform control to cause the notification unit 6 to provide a notification that the tabletop unit-installed part 2 is interfering with the irradiation field, based on the acquired irradiation field range information and member position information. Specifically, the control unit 5 is configured to cause the display unit 60 to display interference information 90 indicating that the tabletop unit-installed part 2 is interfering with the irradiation field, based on the irradiation field range information and the member position information. More specifically, the control unit 5 is configured to cause the display unit 60 to display an interference information image 92 as the interference information 90, which indicates the range where the tabletop unit-installed part 2 interferes in the irradiation field, superimposed on the optical image 40.

The control unit 5 includes a CPU (Central Processing Unit), ROM (Read Only Memory), RAM (Random Access Memory), a GPU (Graphics Processing Unit), or an FPGA (Field-Programmable Gate Array) configured for image processing, and the like.

As shown in FIG. 1, the operation unit 7 is provided in the vicinity of the first X-ray detection unit 32. The user can perform various operations related to X-ray imaging and fluoroscopic imaging using the operation unit 7. In the present embodiment, the X-ray imaging apparatus 100 is a so-called patient-side X-ray imaging apparatus. The operation unit 7 includes, for example, an operation switch group including operation switches for operating the movement of the tabletop 10, and a liquid crystal panel that displays information necessary for imaging the subject 9. Here, a patient-side X-ray imaging apparatus is an X-ray imaging apparatus that allows a user to perform fluoroscopic imaging and the like at a position close to the subject 9 using the operation unit 7.

### Functional Blocks of Control Unit

With reference to FIG. 3, the functional blocks included in the control unit 5 will be described. The control unit 5, which consists of hardware such as a CPU, includes a position information acquisition unit 50 and an image generation unit 51 as functional blocks of software (program). The control unit 5 functions as the position information acquisition unit 50 and the image generation unit 51 by executing a program stored in a storage unit (not shown). The position information acquisition unit 50 and the image generation unit 51 may be individually configured by hardware by providing dedicated processors (processing circuits).

### Position Information Acquisition Unit

The position information acquisition unit 50 is configured to acquire the range of the first irradiation field of X-rays irradiated from the first X-ray irradiation unit 31 and member position information related to the position of the tabletop unit-installed part 2. Specifically, when X-ray imaging or fluoroscopic imaging using the first X-ray imaging unit 30 is selected by the user, the position information acquisition unit 50 is configured to acquire irradiation field range information related to the range of the first irradiation field, and member position information related to the position of the tabletop moving rail 20 and the position of the second X-ray detection unit moving member 21. Note that the range of the first irradiation field includes the range of the X-ray irradiation field when X-rays are actually being irradiated from the first X-ray irradiation unit 31, and the range of the X-ray irradiation field when X-rays would be irradiated from the first X-ray irradiation unit 31.

The position information acquisition unit 50 acquires, for example, position information of the tabletop 10 based on outputs from position sensors such as potentiometers or encoders (not shown) provided in each of a holding member drive unit 83, a first tabletop drive unit 84, and a second tabletop drive unit 85 that drive the tabletop 10. The position information acquisition unit 50 also acquires, for example, position information of the first X-ray irradiation unit 31 and the first X-ray detection unit 32 based on an output from a position sensor such as a potentiometer or an encoder (not shown) provided in a second support column drive unit 87. The position information acquisition unit 50 also acquires, for example, dimensional information of an aperture through which X-rays pass, formed by the shielding members of the collimator 31b in the first X-ray irradiation unit 31, based on a user's operation input to the operation unit 7.

The position information acquisition unit 50 acquires irradiation field range information related to the range of the first irradiation field based on the position information of the tabletop 10, the position information of the first X-ray irradiation unit 31 and the first X-ray detection unit 32, and the dimensional information of the aperture of the collimator 31b in the first X-ray irradiation unit 31.

The position information acquisition unit 50 also acquires, for example, member position information related to the position of the tabletop moving rail 20 based on the acquired position information of the tabletop 10. The position information acquisition unit 50 also acquires, for example, member position information related to the position of the second X-ray detection unit moving member 21 based on an output from a position sensor such as a potentiometer or an encoder (not shown) provided in a second X-ray detection unit drive unit 86 that moves the second X-ray detection unit moving member 21.

### Image Generation Unit

As shown in FIG. 4 to FIG. 7, when X-ray imaging or fluoroscopic imaging using the first X-ray imaging unit 30 is selected by the user, the image generation unit 51 is configured to generate a first superimposed image 91 and to cause the display unit 60 of the notification unit 6 to display the generated first superimposed image 91. The first superimposed image 91 is an image in which an irradiation field image 93 showing a range 99 of the first irradiation field at a predetermined height position, an image showing the position of the tabletop unit-installed part 2, and an interference information image 92 as interference information 90 showing a range where the tabletop unit-installed part 2 interferes in the first irradiation field are superimposed on an optical image 40 captured by the optical imaging unit 4. That is, the image generation unit 51 is configured to provide a notification that the tabletop unit-installed part 2 is interfering with the first irradiation field by causing the display unit 60 to display the interference information image 92 as the interference information 90 included in the first superimposed image 91.

When X-ray imaging or fluoroscopic imaging using the first X-ray imaging unit 30 is selected by the user, the image generation unit 51 acquires the optical image 40 captured by the optical imaging unit 4. The image generation unit 51 also acquires angle of view information of the optical image 40 from the optical imaging unit 4 and acquires position information of the optical imaging unit 4 based on the position information of the first X-ray detection unit 32.

The image generation unit 51 is configured to acquire the range 99 of the first irradiation field at a predetermined height position from the placement surface 10a of the tabletop 10 based on the irradiation field range information related to the range of the first irradiation field, and to generate an irradiation field image 93 showing the acquired range 99 of the first irradiation field at the predetermined height position. The irradiation field image 93 showing the range 99 of the first irradiation field at the predetermined height position is, for example, a frame image showing the outer edge of the range 99 of the first irradiation field at the predetermined height position.

The predetermined height position from the placement surface 10a of the tabletop 10 is, for example, a height position 20 cm away from the placement surface 10a of the tabletop 10 in the Z1 direction. The predetermined height position from the placement surface 10a of the tabletop 10 is not limited to a height position 20 cm away from the placement surface 10a of the tabletop 10 in the Z1 direction, and may be a height position 10 cm away from the placement surface 10a of the tabletop 10 in the Z1 direction, a height position 30 cm away from the placement surface 10a of the tabletop 10 in the Z1 direction, or the position of the placement surface 10a of the tabletop 10, which is a position 0 cm away from the placement surface 10a of the tabletop 10 in the Z1 direction, and is not particularly limited.

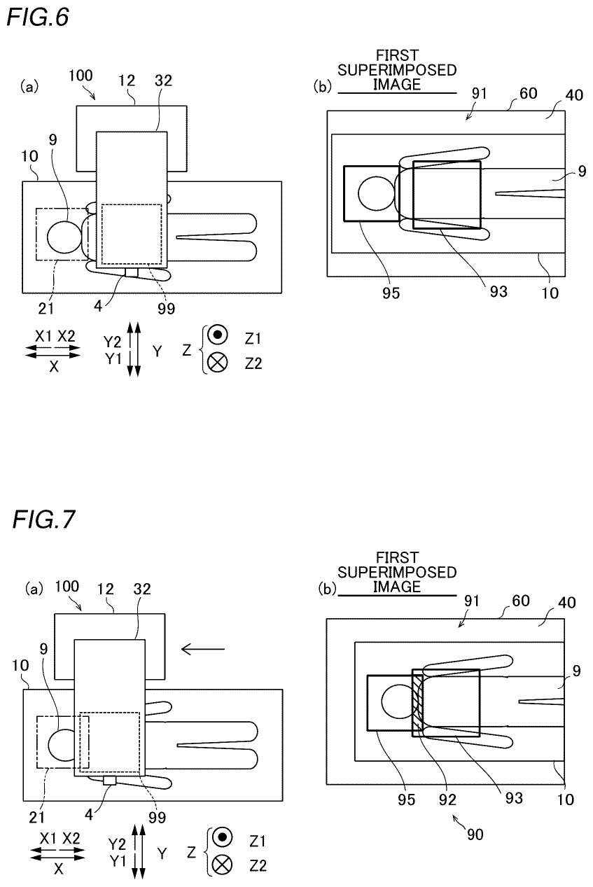

The image generation unit 51 is configured to generate an image showing the position of the tabletop unit-installed part 2 based on the member position information related to the position of the tabletop unit-installed part 2. That is, as shown in FIG. 4(b), the image generation unit 51 generates an image 94 showing the position of the tabletop moving rail 20 based on the member position information related to the position of the tabletop moving rail 20, and as shown in FIG. 6(b), generates an image 95 showing the position of the second X-ray detection unit moving member 21 based on the member position information related to the position of the second X-ray detection unit moving member 21. The image 94 showing the position of the tabletop moving rail 20 is, for example, a frame image showing the outer edge of the tabletop moving rail 20. The image 95 showing the position of the second X-ray detection unit moving member 21 is, for example, a frame image showing the outer edge of the second X-ray detection unit moving member 21. Note that the color of the frame image showing the outer edge of the range of the first irradiation field, the color of the frame image showing the outer edge of the tabletop moving rail 20, and the color of the frame image showing the outer edge of the second X-ray detection unit moving member 21 are represented by different colors.

Furthermore, as shown in FIG. 5(b) and FIG. 7(b), the image generation unit 51 is configured to generate an interference information image 92 as interference information 90 showing a range where the tabletop unit-installed part 2 interferes in the first irradiation field, based on the irradiation field range information related to the range of the first irradiation field and the member position information.

That is, as shown in FIG. 5(b), the image generation unit 51 generates the interference information image 92 as the interference information 90 showing the range where the tabletop moving rail 20 interferes in the first irradiation field, based on the irradiation field range information related to the range of the first irradiation field and the member position information related to the position of the tabletop moving rail 20. The interference information image 92 is, for example, an image showing the range where the tabletop moving rail 20 interferes in the first irradiation field, with a semi-transparent color that allows the optical image 40 to be visible.

Furthermore, as shown in FIG. 7(b), the image generation unit 51 generates the interference information image 92 as the interference information 90 showing the range where the second X-ray detection unit moving member 21 interferes in the first irradiation field, based on the irradiation field range information related to the range of the first irradiation field and the member position information related to the position of the second X-ray detection unit moving member 21. The interference information image 92 is, for example, an image showing the range where the second X-ray detection unit moving member 21 interferes in the first irradiation field, with a semi-transparent color that allows the optical image 40 to be visible.

Note that since the X-rays irradiated from the X-ray tube 31a of the first X-ray irradiation unit 31 and passing through the aperture formed by the shielding members of the collimator 31b diffuse and spread, the range of the first irradiation field at the height position of the tabletop moving rail 20 and the range 99 of the first irradiation field at the predetermined height position from the placement surface 10a of the tabletop 10 are different. The image generation unit 51 generates the interference information image 92 as the interference information 90 showing the range where the tabletop moving rail 20 interferes in the first irradiation field at the height position of the tabletop moving rail 20 located between the first X-ray irradiation unit 31 and the first X-ray detection unit 32. Similarly, the range of the first irradiation field at the height position of the second X-ray detection unit moving member 21 and the range 99 of the first irradiation field at the predetermined height position from the placement surface 10a of the tabletop 10 are different. The image generation unit 51 generates the interference information image 92 as the interference information 90 showing the range where the second X-ray detection unit moving member 21 interferes in the first irradiation field at the height position of the second X-ray detection unit moving member 21 located between the first X-ray irradiation unit 31 and the first X-ray detection unit 32.

As shown in FIG. 4(b) and FIG. 6(b), the image generation unit 51 generates a first superimposed image 91 in which the irradiation field image 93 showing the range 99 of the first irradiation field at the predetermined height position and the image showing the position of the tabletop unit-installed part 2 are aligned and superimposed on the optical image 40, and causes the display unit 60 to display the generated first superimposed image 91. Furthermore, in a state where the tabletop unit-installed part 2 interferes in the first irradiation field, as shown in FIG. 5(b) and FIG. 7(b), the image generation unit 51 generates a first superimposed image 91 in which the irradiation field image 93 showing the range 99 of the first irradiation field at the predetermined height position, the image showing the position of the tabletop unit-installed part 2, and the interference information image 92 as the interference information 90 showing the range where the tabletop unit-installed part 2 interferes in the first irradiation field are aligned and superimposed on the optical image 40, and causes the display unit 60 to display the generated first superimposed image 91.

### Control of Display of Interference Information Image by Control Unit

### Control of Display of Interference Information Image Related to Tabletop Moving Rail in X-Ray Imaging or Fluoroscopic Imaging Using First X-Ray Imaging Unit

With reference to FIG. 4 and FIG. 5, the control of the display of the interference information image 92 related to the tabletop moving rail 20 in X-ray imaging or fluoroscopic imaging using the first X-ray imaging unit 30 by the control unit 5 will be described.

The example shown in FIG. 4 and FIG. 5 is an example of the control of the display of the interference information image 92 related to the tabletop moving rail 20 when the first X-ray imaging unit 30 is moved in the Y1 direction during fluoroscopic imaging of the subject 9 in a supine position on the tabletop 10. Note that FIG. 4(a) and FIG. 5(a) are views of the tabletop 10 from the Z1 direction. For convenience of explanation, the image 95 showing the position of the second X-ray detection unit moving member 21 is omitted in FIG. 4(b) and FIG. 5(b).

In both the states of FIG. 4(a) and FIG. 5(a), the optical imaging unit 4 captures an optical image 40 of the subject 9 placed on the tabletop 10 and the tabletop unit 1. The image generation unit 51 acquires the optical image 40 captured by the optical imaging unit 4. The image generation unit 51 also acquires the irradiation field range information related to the range of the first irradiation field and the member position information related to the position of the tabletop moving rail 20, which were acquired by the position information acquisition unit 50.

FIG. 4(a) is a schematic diagram showing the positional relationship among the subject 9, the first X-ray imaging unit 30, the tabletop 10, and the tabletop moving rail 20 before moving the first X-ray imaging unit 30 in the Y1 direction. In FIG. 4(a), the tabletop moving rail 20, which is not visible to the user because it is disposed below the tabletop 10, is represented by a dash-dotted line. FIG. 4(b) is a schematic diagram of the first superimposed image 91 displayed on the display unit 60 before moving the first X-ray imaging unit 30 in the Y1 direction. In the states of FIG. 4(a) and FIG. 4(b), the tabletop moving rail 20 does not interfere with the first irradiation field.

In the state of FIG. 4(a), the image generation unit 51 acquires the range 99 of the first irradiation field at the predetermined height position from the placement surface 10a of the tabletop 10 based on the irradiation field range information related to the range of the first irradiation field, and generates a frame image showing the acquired range 99 of the first irradiation field at the predetermined height position. The image generation unit 51 also generates a frame image showing the position of the tabletop moving rail 20 based on the member position information related to the position of the tabletop moving rail 20. Note that the image generation unit 51 does not generate the interference information image 92 because the tabletop moving rail 20 does not interfere in the first irradiation field, based on the irradiation field range information related to the range of the first irradiation field and the member position information related to the position of the tabletop moving rail 20. Then, as shown in FIG. 4(b), the image generation unit 51 causes the display unit 60 to display the first superimposed image 91 in which the frame image showing the range of the first irradiation field and the frame image showing the position of the tabletop moving rail 20 are superimposed on the optical image 40.

FIG. 5(a) is a schematic diagram showing the positional relationship among the subject 9, the first X-ray imaging unit 30, the tabletop 10, and the tabletop moving rail 20 after moving the first X-ray imaging unit 30 in the Y1 direction. Also in FIG. 5(a), the tabletop moving rail 20, which is not visible to the user because it is disposed below the tabletop 10, is represented by a dash-dotted line. FIG. 5(b) is a schematic diagram of the first superimposed image 91 displayed on the display unit 60 after moving the first X-ray imaging unit 30 in the Y1 direction. In the states of FIG. 5(a) and FIG. 5(b), the tabletop moving rail 20 interferes with the first irradiation field.

In the state of FIG. 5(a), similarly to the state of FIG. 4(a), the image generation unit 51 generates a frame image showing the range 99 of the first irradiation field at the predetermined height position and generates a frame image showing the position of the tabletop moving rail 20. Here, the image generation unit 51 generates the interference information image 92 because the tabletop moving rail 20 interferes in the first irradiation field, based on the irradiation field range information related to the range of the first irradiation field and the member position information related to the position of the tabletop moving rail 20. The interference information image 92 is, as an example, an image showing the range where the tabletop moving rail 20 interferes in the first irradiation field, with a semi-transparent color that allows the optical image 40 to be visible. Then, as shown in FIG. 5(b), the image generation unit 51 causes the display unit 60 to display the first superimposed image 91 in which the frame image showing the range of the first irradiation field, the frame image showing the position of the tabletop moving rail 20, and the interference information image 92 are superimposed on the optical image 40.

### Control of Display of Interference Information Image Related to Second X-Ray Detection Unit Moving Member in X-Ray Imaging or Fluoroscopic Imaging Using First X-Ray Imaging Unit

With reference to FIG. 6 and FIG. 7, the control of the display of the interference information image 92 related to the second X-ray detection unit moving member 21 in X-ray imaging or fluoroscopic imaging using the first X-ray imaging unit 30 by the control unit 5 will be described.

The example shown in FIG. 6 and FIG. 7 is an example of the control of the display of the interference information image 92 related to the second X-ray detection unit moving member 21 when the first X-ray imaging unit 30 is moved in the X1 direction during fluoroscopic imaging of the subject 9 in a supine position on the tabletop 10. Note that FIG. 6(a) and FIG. 7(a) are views of the tabletop 10 from the Z1 direction. For convenience of explanation, the image 94 showing the position of the tabletop moving rail 20 is omitted in FIG. 6(b) and FIG. 7(b).

In both the states of FIG. 6(a) and FIG. 7(a), the optical imaging unit 4 captures an optical image 40 of the subject 9 placed on the tabletop 10 and the tabletop unit 1. The image generation unit 51 acquires the optical image 40 captured by the optical imaging unit 4. The image generation unit 51 also acquires the irradiation field range information related to the range of the first irradiation field and the member position information related to the position of the second X-ray detection unit moving member 21, which were acquired by the position information acquisition unit 50.

FIG. 6(a) is a schematic diagram showing the positional relationship among the subject 9, the first X-ray imaging unit 30, the tabletop 10, and the second X-ray detection unit moving member 21 before moving the first X-ray imaging unit 30 in the X1 direction. In FIG. 6(a), the second X-ray detection unit moving member 21, which is not visible to the user because it is disposed below the tabletop 10, is represented by a two-dot chain line. FIG. 6(b) is a schematic diagram of the first superimposed image 91 displayed on the display unit 60 before moving the first X-ray imaging unit 30 in the X1 direction. In the states of FIG. 6(a) and FIG. 6(b), the second X-ray detection unit moving member 21 does not interfere with the first irradiation field.

In the state of FIG. 6(a), the image generation unit 51 acquires the range 99 of the first irradiation field at the predetermined height position from the placement surface 10a of the tabletop 10 based on the irradiation field range information related to the range of the first irradiation field, and generates a frame image showing the acquired range 99 of the first irradiation field at the predetermined height position. The image generation unit 51 also generates a frame image showing the position of the second X-ray detection unit moving member 21 based on the member position information related to the position of the second X-ray detection unit moving member 21. Note that the image generation unit 51 does not generate the interference information image 92 because the second X-ray detection unit moving member 21 does not interfere in the first irradiation field, based on the irradiation field range information related to the range of the first irradiation field and the member position information related to the position of the second X-ray detection unit moving member 21. Then, as shown in FIG. 6(b), the image generation unit 51 causes the display unit 60 to display the first superimposed image 91 in which the frame image showing the range of the first irradiation field and the frame image showing the position of the second X-ray detection unit moving member 21 are superimposed on the optical image 40.

FIG. 7(a) is a schematic diagram showing the positional relationship among the subject 9, the first X-ray imaging unit 30, the tabletop 10, and the second X-ray detection unit moving member 21 after moving the first X-ray imaging unit 30 in the X1 direction. Also in FIG. 7(a), the second X-ray detection unit moving member 21, which is not visible to the user because it is disposed below the tabletop 10, is represented by a two-dot chain line. FIG. 7(b) is a schematic diagram of the first superimposed image 91 displayed on the display unit 60 after moving the first X-ray imaging unit 30 in the X1 direction. In the states of FIG. 7(a) and FIG. 7(b), the second X-ray detection unit moving member 21 interferes with the first irradiation field.

In the state of FIG. 7(a), similarly to the state of FIG. 6(a), the image generation unit 51 generates a frame image showing the range 99 of the first irradiation field at the predetermined height position and generates a frame image showing the position of the second X-ray detection unit moving member 21. Here, the image generation unit 51 generates the interference information image 92 because the second X-ray detection unit moving member 21 interferes in the first irradiation field, based on the irradiation field range information related to the range of the first irradiation field and the member position information related to the position of the second X-ray detection unit moving member 21. The interference information image 92 is, as an example, an image showing the range where the second X-ray detection unit moving member 21 interferes in the first irradiation field, with a semi-transparent color that allows the optical image 40 to be visible. Then, as shown in FIG. 7(b), the image generation unit 51 causes the display unit 60 to display the first superimposed image 91 in which the frame image showing the range of the first irradiation field, the frame image showing the position of the second X-ray detection unit moving member 21, and the interference information image 92 are superimposed on the optical image 40.

### Effects of First Embodiment

In the first embodiment, the following effects can be obtained.

In the first embodiment, as described above, the control unit 5 performs control to cause the notification unit 6 to provide a notification that the tabletop unit-installed part 2 is interfering with the first irradiation field, based on the irradiation field range information related to the range of the irradiation field of X-rays irradiated from the first X-ray irradiation unit 31 and the member position information related to the position of the tabletop unit-installed part 2. Thereby, the user can recognize that the tabletop unit-installed part 2 is interfering with the first irradiation field because the notification unit 6 provides the notification that the tabletop unit-installed part 2 is interfering with the first irradiation field. Therefore, the user can eliminate the interference of the tabletop unit-installed part 2 with the first irradiation field. As a result, it is possible to suppress an increase in radiation exposure dose due to retaking of X-ray images or extension of fluoroscopic imaging time caused by interference of the tabletop unit-installed part 2 with the first irradiation field.

Furthermore, in the first embodiment, by configuring as follows, the following further effects can be obtained.

That is, in the first embodiment, as described above, the tabletop unit-installed part 2 includes the tabletop moving rail 20, which is provided at both ends in the transverse direction of the tabletop unit 1 and slides and moves the tabletop 10 included in the tabletop unit 1 in the longitudinal direction of the tabletop unit 1, and the second X-ray detection unit moving member 21, which is provided on the tabletop unit 1, holds the second X-ray detection unit 35 placed on the tabletop unit 1, and moves the second X-ray detection unit 35 in the longitudinal direction of the tabletop unit 1, and is configured to include a metal part with low X-ray transmittance. Thereby, since the user can recognize that the tabletop moving rail 20 and the second X-ray detection unit moving member 21 including the metal part are interfering with the first irradiation field, the user can easily eliminate the interference of the tabletop moving rail 20 and the second X-ray detection unit moving member 21 with the first irradiation field. As a result, it is possible to easily suppress an increase in radiation exposure dose due to retaking of X-ray images or extension of fluoroscopic imaging time caused by interference of the tabletop moving rail 20 and the second X-ray detection unit moving member 21 including the metal part with the first irradiation field.

Furthermore, in the first embodiment, as described above, the notification unit 6 includes the display unit 60, and the control unit 5 is configured to cause the display unit 60 to display the interference information 90 indicating that the tabletop unit-installed part 2 is interfering with the first irradiation field, based on the irradiation field range information and the member position information. Thereby, since the interference information 90 indicating that the tabletop unit-installed part 2 is interfering with the first irradiation field is displayed on the display unit 60, the user can visually and easily recognize that the tabletop unit-installed part 2 is interfering with the first irradiation field.

Furthermore, in the first embodiment, as described above, the apparatus further includes the optical imaging unit 4 that captures the optical image 40 of the subject 9 and the tabletop unit 1, the display unit 60 is configured to display the optical image 40 captured by the optical imaging unit 4, and the control unit 5 is configured to cause the display unit 60 to display the interference information image 92 as the interference information 90, which indicates the range where the tabletop unit-installed part 2 interferes in the first irradiation field, superimposed on the optical image 40. Thereby, since the interference information image 92 indicating that the tabletop unit-installed part 2 is interfering with the first irradiation field is displayed on the display unit 60, the user can more easily recognize that the tabletop unit-installed part 2 is interfering with the first irradiation field.

Furthermore, in the first embodiment, as described above, the X-ray irradiation unit includes the first X-ray irradiation unit 31 and the second X-ray irradiation unit 34, the X-ray detection unit includes the first X-ray detection unit 32 and the second X-ray detection unit 35, the X-ray imaging unit 3 includes the first X-ray imaging unit 30, which includes the first X-ray irradiation unit 31 disposed on the tabletop unit 1 and the first X-ray detection unit 32 disposed at a position facing the first X-ray irradiation unit 31 across the tabletop 10, and the second X-ray imaging unit 33, which includes the second X-ray detection unit 35 disposed on the tabletop unit 1 and the second X-ray irradiation unit 34 disposed on the side opposite to the second X-ray detection unit 35 across the tabletop 10, the tabletop unit-installed part 2 is the tabletop moving rail 20, and the control unit 5 is configured to cause the notification unit 6 to provide a notification that the tabletop moving rail 20 is interfering with the first irradiation field, based on the irradiation field range information related to the range of the first irradiation field of X-rays irradiated from the first X-ray irradiation unit 31 and the member position information related to the position of the tabletop moving rail 20. Thereby, since X-rays are irradiated to the subject 9 through the tabletop 10 by the first X-ray irradiation unit 31 of the first X-ray imaging unit 30, even in a configuration where the irradiation field on the body surface of the subject 9 cannot be confirmed by an irradiation field lamp, the user can recognize that the tabletop moving rail 20 is interfering with the first irradiation field. Therefore, even with a configuration where X-rays are irradiated to the subject 9 through the tabletop 10 by the first X-ray irradiation unit 31 of the first X-ray imaging unit 30, it is possible to suppress an increase in radiation exposure dose due to retaking of X-ray images or extension of fluoroscopic imaging time caused by interference of the tabletop moving rail 20 with the first irradiation field.

Furthermore, in the first embodiment, as described above, the X-ray irradiation unit includes the first X-ray irradiation unit 31 and the second X-ray irradiation unit 34, the X-ray detection unit includes the first X-ray detection unit 32 and the second X-ray detection unit 35, the X-ray imaging unit 3 includes the first X-ray imaging unit 30, which includes the first X-ray irradiation unit 31 disposed on the tabletop unit 1 and the first X-ray detection unit 32 disposed at a position facing the first X-ray irradiation unit 31 across the tabletop 10, and the second X-ray imaging unit 33, which includes the second X-ray detection unit 35 disposed on the tabletop unit 1 and the second X-ray irradiation unit 34 disposed on the side opposite to the second X-ray detection unit 35 across the tabletop 10, the tabletop unit-installed part 2 is the second X-ray detection unit moving member 21 as the X-ray detection unit moving member, and the control unit 5 is configured to cause the notification unit 6 to provide a notification that the second X-ray detection unit moving member 21 is interfering with the first irradiation field, based on the irradiation field range information related to the range of the first irradiation field of X-rays irradiated from the first X-ray irradiation unit 31 and the member position information related to the position of the second X-ray detection unit moving member 21. Thereby, since X-rays are irradiated to the subject 9 through the tabletop 10 by the first X-ray irradiation unit 31 of the first X-ray imaging unit 30, even in a configuration where the irradiation field on the body surface of the subject 9 cannot be confirmed by an irradiation field lamp, the user can recognize that the second X-ray detection unit moving member 21 is interfering with the first irradiation field. Therefore, even with a configuration where X-rays are irradiated to the subject 9 through the tabletop 10 by the first X-ray irradiation unit 31 of the first X-ray imaging unit 30, it is possible to suppress an increase in radiation exposure dose due to retaking of X-ray images or extension of fluoroscopic imaging time caused by interference of the second X-ray detection unit moving member 21 with the first irradiation field.

Furthermore, in the first embodiment, as described above, the apparatus is configured to cause the notification unit 6 to provide a notification that the tabletop unit-installed part 2 is interfering with the first irradiation field based on the irradiation field range information and the member position information at least during fluoroscopic imaging by the first X-ray imaging unit 30. Thereby, since the notification unit 6 provides the notification that the tabletop unit-installed part 2 is interfering with the first irradiation field at least during fluoroscopic imaging, it is possible to easily suppress an increase in radiation exposure dose due to extension of fluoroscopic imaging time caused by interference of the tabletop unit-installed part 2 with the first irradiation field.

### Second Embodiment

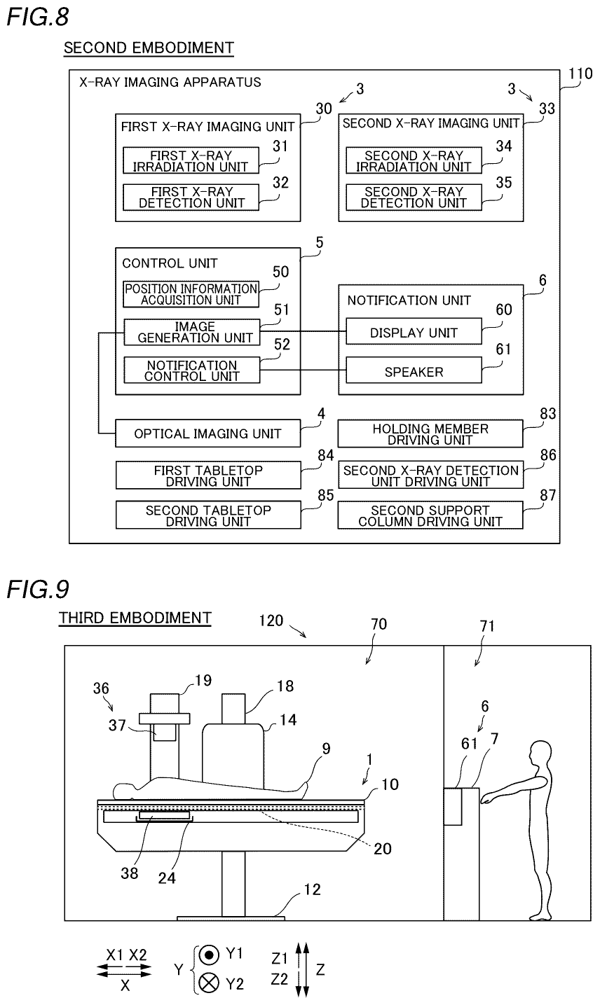

With reference to FIG. 8, a second embodiment will be described. In this second embodiment, an example will be described in which, unlike the first embodiment where the notification unit 6 includes the display unit 60 and the control unit 5 is configured to cause the display unit 60 to display the first superimposed image 91 in which the interference information image 92 is superimposed on the optical image 40, the notification unit 6 includes a speaker 61, and the control unit 5 is configured to cause the speaker 61 to provide a notification that the tabletop unit-installed part 2 is interfering with the X-ray irradiation field. Note that the same components as in the first embodiment are denoted by the same reference numerals, and a description thereof is omitted.

### Configuration of X-Ray Imaging Apparatus

The configuration of an X-ray imaging apparatus 110 according to the present embodiment will be described.

The notification unit 6 includes a speaker 61. The speaker 61 is configured to provide a notification that the tabletop unit-installed part 2 is interfering with the first irradiation field under the control of the control unit 5. Specifically, the speaker 61 is configured to provide a notification that the tabletop unit-installed part 2 is interfering with the first irradiation field by outputting a notification sound.

The control unit 5 is configured to cause the speaker 61 to provide a notification that the tabletop unit-installed part 2 is interfering with the X-ray irradiation field, based on the irradiation field range information and the member position information.

### Functional Blocks of Control Unit

The functional blocks included in the control unit 5 will be described. The control unit 5, which consists of hardware such as a CPU, includes a position information acquisition unit 50, an image generation unit 51, and a notification control unit 52 as functional blocks of software (program). The control unit 5 functions as the position information acquisition unit 50, the image generation unit 51, and the notification control unit 52 by executing a program stored in a storage unit (not shown). The position information acquisition unit 50, the image generation unit 51, and the notification control unit 52 may be individually configured by hardware by providing dedicated processors (processing circuits). Note that the position information acquisition unit 50 has the same configuration as the position information acquisition unit 50 in the first embodiment, so a description thereof is omitted here.

When X-ray imaging or fluoroscopic imaging using the first X-ray imaging unit 30 is selected by the user, the image generation unit 51 is configured to cause the display unit 60 of the notification unit 6 to display an irradiation field superimposed image in which an irradiation field image 93 (see FIG. 4(a)) showing a range 99 of the first irradiation field at a predetermined height position is superimposed on an optical image 40 (see FIG. 4(a)) captured by the optical imaging unit 4. When X-ray imaging or fluoroscopic imaging using the first X-ray imaging unit 30 is selected by the user, the image generation unit 51 acquires the optical image 40 captured by the optical imaging unit 4.

The image generation unit 51 is configured to acquire the range 99 of the first irradiation field at the predetermined height position from the placement surface 10a of the tabletop 10 based on the irradiation field range information related to the range of the first irradiation field, and to generate an irradiation field image 93 showing the acquired range 99 of the first irradiation field at the predetermined height position. Since the irradiation field image 93 showing the range 99 of the first irradiation field at the predetermined height position is the same as in the first embodiment, a description thereof is omitted here.

The image generation unit 51 generates an irradiation field superimposed image in which the irradiation field image 93 showing the range 99 of the first irradiation field at the predetermined height position is aligned and superimposed on the optical image 40, and causes the display unit 60 to display the generated irradiation field superimposed image. That is, in the second embodiment, the image generation unit 51 does not generate the interference information image 92 as the interference information 90 showing the range where the tabletop unit-installed part 2 interferes in the first irradiation field.

When X-ray imaging or fluoroscopic imaging using the first X-ray imaging unit 30 is selected by the user, the notification control unit 52 is configured to acquire irradiation field range information related to the range of the first irradiation field of X-rays irradiated from the first X-ray irradiation unit 31 and member position information related to the position of the tabletop unit-installed part 2. The notification control unit 52 is also configured to perform control to cause the speaker 61 to provide a notification that the tabletop unit-installed part 2 is interfering with the first irradiation field, based on the acquired irradiation field range information and member position information.

The notification control unit 52 acquires, from the position information acquisition unit 50, the irradiation field range information related to the range of the first irradiation field, and the member position information related to the position of the tabletop moving rail 20 and the position of the second X-ray detection unit moving member 21. The notification control unit 52 also causes the speaker 61 to provide a notification that the tabletop moving rail 20 is interfering in the first irradiation field, based on the acquired irradiation field range information related to the range of the first irradiation field and the member position information related to the position of the tabletop moving rail 20. The notification control unit 52 also causes the speaker 61 to provide a notification that the second X-ray detection unit moving member 21 is interfering in the first irradiation field, based on the acquired irradiation field range information related to the range of the first irradiation field and the member position information related to the position of the second X-ray detection unit moving member 21.

The notification control unit 52 is configured to provide a notification by outputting a notification sound from the speaker 61. That is, the notification control unit 52 is configured to provide a notification that the tabletop unit-installed part 2 is interfering with the first irradiation field by outputting a notification sound from the speaker 61. The notification sound may be a warning sound or a warning message by voice. As a voice warning message, for example, information indicating that the tabletop unit-installed part 2 is interfering with the first irradiation field, such as “The tabletop moving rail 20 is in the irradiation field,” may be output, or information prompting the movement of the first X-ray imaging unit 30 or the tabletop 10, such as “The tabletop moving rail 20 is interfering with the irradiation field, so please move the first X-ray imaging unit 30 in the XX direction of the tabletop 10,” may be output.

Other configurations of the second embodiment are the same as those of the first embodiment.

### Effects of Second Embodiment

In the second embodiment, the following effects can be obtained.

In the second embodiment, as described above, the notification unit 6 includes the speaker 61, and the control unit 5 is configured to cause the speaker 61 to provide a notification that the tabletop unit-installed part 2 is interfering with the irradiation field, based on the irradiation field range information and the member position information. Thereby, since the notification that the tabletop unit-installed part 2 is interfering with the first irradiation field is provided by the speaker 61, the user can audibly and easily recognize that the tabletop unit-installed part 2 is interfering with the first irradiation field.

Other effects of the second embodiment are the same as those of the first embodiment.

### Third Embodiment

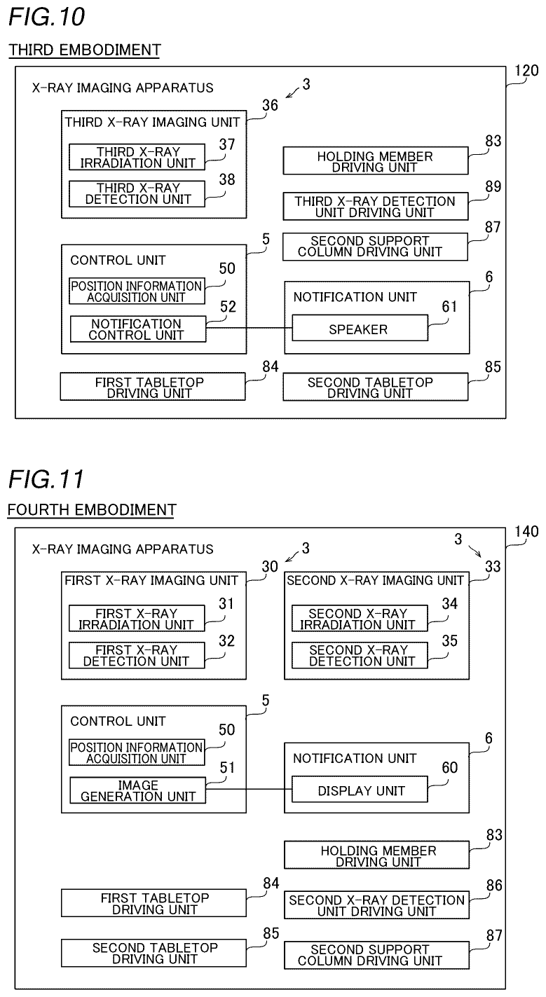

With reference to FIG. 9 and FIG. 10, a third embodiment will be described. In this third embodiment, an example will be described in which, unlike the second embodiment where the first X-ray imaging unit 30 includes the first X-ray irradiation unit 31 disposed on the tabletop unit 1 and the first X-ray detection unit 32 disposed at a position facing the first X-ray irradiation unit 31 across the tabletop 10, and the operation unit 7 is provided in the vicinity of the first X-ray detection unit 32, a third X-ray imaging unit 36 includes a third X-ray detection unit 38 disposed on the tabletop unit 1 and a third X-ray irradiation unit 37 disposed on the side opposite to the third X-ray detection unit 38 across the tabletop 10, and the X-ray imaging apparatus 120 is placed in an examination room 70, and the operation unit 7 is provided in an operation room 71 adjacent to the examination room 70. Note that the same components as in the second embodiment are denoted by the same reference numerals, and a description thereof is omitted.

### Configuration of X-Ray Imaging Apparatus

The configuration of an X-ray imaging apparatus 120 according to the present embodiment will be described.

As shown in FIG. 9, the apparatus includes a tabletop unit 1, a tabletop unit-installed part 2, a third X-ray imaging unit 36, a control unit 5 (see FIG. 10), a notification unit 6, and an operation unit 7. In the X-ray imaging apparatus 120, the tabletop unit 1 and the third X-ray imaging unit 36 are placed in an examination room 70, and the notification unit 6 and the operation unit 7 are placed in an operation room 71 adjacent to the examination room 70. In the present embodiment, the X-ray imaging apparatus 120 is a so-called remote-controlled X-ray imaging apparatus. In the present embodiment, an optical imaging unit 4 is not provided. Here, a remote-controlled X-ray imaging apparatus is an X-ray imaging apparatus in which an examination room 70 and an operation room 71 are provided separately, and a user can perform fluoroscopic imaging and the like in the operation room 71 using the operation unit 7.

The tabletop unit 1 includes a base 12, a third support column 18 provided on the base 12, a holding member 14 provided to be vertically movable with respect to the third support column 18, and a tabletop 10 rotatably connected to the holding member 14. The holding member 14 moves up and down in the Z direction.

The tabletop unit-installed part 2 includes a tabletop moving rail 20. The tabletop moving rail 20 is provided at both ends in the transverse direction of the tabletop unit 1. The tabletop moving rail 20 is provided below the tabletop 10. The tabletop moving rail 20 is configured to engage with an engaging part provided on the surface of the tabletop 10 opposite to the placement surface 10a, and to slide and move the tabletop 10 in the longitudinal direction (X direction) of the tabletop unit 1. The tabletop moving rail 20 is formed of a metal material. The tabletop 10 is configured to slide and move in the longitudinal direction (X direction) of the tabletop unit 1 by a first tabletop drive unit 84 via the tabletop moving rail 20. The tabletop 10 is configured to be movable relative to the third X-ray imaging unit 36.

The third X-ray imaging unit 36 includes a third X-ray irradiation unit 37, a third X-ray detection unit 38, and a fourth support column 19. The third X-ray irradiation unit 37 is supported by the fourth support column 19. The third X-ray detection unit 38 is disposed on the side (Z2 side) opposite to the placement surface 10a of the tabletop 10. The fourth support column 19 including the third X-ray irradiation unit 37 and the third X-ray detection unit 38 reciprocate in synchronization with each other in the longitudinal direction of the tabletop 10. The third X-ray imaging unit 36 is configured to be movable relative to the tabletop 10. Note that the third X-ray imaging unit 36, the third X-ray irradiation unit 37, and the third X-ray detection unit 38 are examples of the “X-ray imaging unit,” “X-ray irradiation unit,” and “X-ray detection unit,” respectively, in the claims.

Furthermore, a third X-ray detection unit moving member 24 holds the third X-ray detection unit 38 placed on the tabletop unit 1. The third X-ray detection unit moving member 24 is configured to move in the longitudinal direction (X direction) of the tabletop unit 1 below the tabletop 10, together with the held third X-ray detection unit 38. The third X-ray detection unit moving member 24 is moved in the longitudinal direction (X direction) of the tabletop unit 1 by a third X-ray detection unit drive unit 89.

Similarly to the second embodiment, the notification unit 6 includes a speaker 61. The speaker 61 is configured to provide a notification that the tabletop moving rail 20 is interfering with a third irradiation field under the control of the control unit 5. Specifically, the speaker 61 is configured to provide a notification that the tabletop moving rail 20 is interfering with the third irradiation field by outputting a notification sound. The control unit 5 is also configured to cause the speaker 61 to provide a notification that the tabletop moving rail 20 is interfering with the third irradiation field, based on the irradiation field range information and the member position information.

Other configurations of the third embodiment are the same as those of the second embodiment.

### Effects of Third Embodiment

The third embodiment can also achieve the same effects as the second embodiment.

### Fourth Embodiment

With reference to FIG. 11 to FIG. 15, a fourth embodiment will be described. In this fourth embodiment, an example will be described in which, unlike the first embodiment where the control unit 5 causes the display unit 60 to display the interference information image 92 as the interference information 90 in the first irradiation field superimposed on the optical image 40, the control unit 5 is configured to cause the display unit 60 to display an interference information image 192 as the interference information 90 superimposed on an irradiation field image 193 including an X-ray image. Note that the same components as in the first embodiment are denoted by the same reference numerals, and a description thereof is omitted.

### Overview

In an X-ray imaging apparatus 140, when fluoroscopic imaging is performed on a desired region (region of interest) of a subject 9 placed on a tabletop 10, and then fluoroscopic imaging is performed on a different region of interest, positioning of the X-ray irradiation field may be performed by moving the tabletop 10 or the X-ray imaging unit 3 after the irradiation of X-rays by the X-ray imaging unit 3 is stopped. The positioning of the X-ray irradiation field is performed by aligning the X-ray irradiation field and the region of interest on the subject 9 using an LIH (Last Image Hold) image 98 displayed on a display unit 60. The LIH image 98 is the last frame image among fluoroscopic images collected by fluoroscopic imaging, and is usually continuously displayed on the display unit 60 even after fluoroscopic imaging has been performed.

Here, it can be said that the image displayed on the entire display unit 60 including the LIH image 98 shows the range of the X-ray irradiation field when X-rays are irradiated at that point in time. Therefore, the positioning of the X-ray irradiation field is performed by moving the tabletop 10 or the X-ray imaging unit 3 by the user's operation of an operation unit 7 while the user visually confirms the LIH image 98 displayed on the display unit 60. When the tabletop 10 or the X-ray imaging unit 3 is moved, the control unit 5 moves the display position of the LIH image 98 displayed on the display unit 60 according to the moving direction and moving distance of the tabletop 10, or the moving direction and moving distance of the X-ray imaging unit 3. That is, the control unit 5 moves the LIH image 98 on the display unit 60 so as to follow the movement of the tabletop 10 or the X-ray imaging unit 3. At this time, depending on the moving direction, moving distance of the tabletop 10 or the X-ray imaging unit 3, and the dimension of the aperture of the collimator 31b, the moved LIH image 98 on the display unit 60 may not be displayed in its entirety, and only a part of it may be displayed on the display unit 60.

If it is determined that the X-ray irradiation field and the region of interest are aligned based on the image on the display unit 60 including the LIH image 98, the positioning of the X-ray irradiation field is completed. Then, fluoroscopic imaging is started by irradiating X-rays from the X-ray irradiation unit. However, after the positioning of the X-ray irradiation field is performed using the LIH image 98 displayed on the display unit 60, there is a possibility that the tabletop unit-installed part 2 is interfering with the range of the X-ray irradiation field. Therefore, by making the user aware that the tabletop unit-installed part 2 is interfering with the X-ray irradiation field, an increase in radiation exposure dose due to retaking of X-ray images or extension of fluoroscopic imaging time caused by interference of the tabletop unit-installed part 2 with the X-ray irradiation field is suppressed.

### Configuration of X-Ray Imaging Apparatus

With reference to FIG. 11 to FIG. 13, the configuration of an X-ray imaging apparatus 140 according to the present embodiment will be described.

The display unit 60 is configured to display an irradiation field image 193 that includes an X-ray image based on a detection signal detected by the first X-ray detection unit 32 and shows the range of the irradiation field. Specifically, when fluoroscopic imaging using the first X-ray imaging unit 30 is selected by the user, the display unit 60 is configured to display an irradiation field image 193 that includes an LIH image 98 based on the detection signal detected by the first X-ray detection unit 32 and shows the range of the irradiation field. The LIH image 98 is an example of the “X-ray image” in the claims.

When fluoroscopic imaging using the first X-ray imaging unit 30 is selected by the user, the display unit 60 continuously displays the LIH image 98 after the fluoroscopic imaging has been performed.

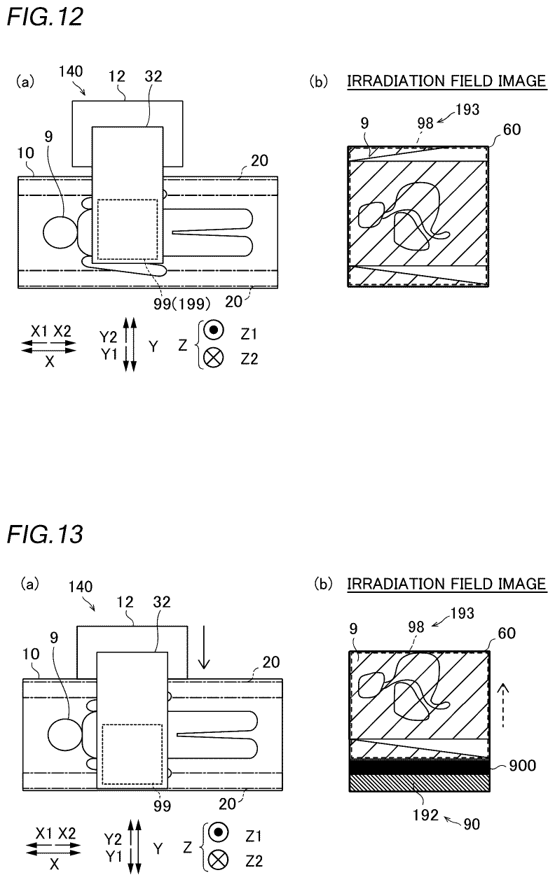

As shown in FIG. 12(b), the control unit 5 causes the display unit 60 to continuously display the LIH image 98 after the fluoroscopic imaging has been performed. As shown in FIG. 13(b), the control unit 5 is also configured to cause the display unit 60 to display an interference information image 192 as the interference information 90, which indicates the range where the tabletop unit-installed part 2 interferes in the first irradiation field, superimposed on the irradiation field image 193 including the X-ray image. Specifically, when fluoroscopic imaging using the first X-ray imaging unit 30 is selected by the user, after the fluoroscopic imaging has been performed, the control unit 5 causes the display unit 60 to display the interference information image 192 as the interference information 90, which indicates the range where the tabletop unit-installed part 2 interferes in the first irradiation field, superimposed on the irradiation field image 193 including the LIH image 98.

### Functional Blocks of Control Unit

The functional blocks included in the control unit 5 will be described. The control unit 5, which consists of hardware such as a CPU, includes a position information acquisition unit 50 and an image generation unit 51 as functional blocks of software (program). The control unit 5 functions as the position information acquisition unit 50 and the image generation unit 51 by executing a program stored in a storage unit (not shown). The position information acquisition unit 50 and the image generation unit 51 may be individually configured by hardware by providing dedicated processors (processing circuits). Note that the position information acquisition unit 50 has the same configuration as the position information acquisition unit 50 in the first embodiment, so a description thereof is omitted here.

### Image Generation Unit

The image generation unit 51 is configured to generate an X-ray image based on a detection signal output from the first X-ray detection unit 32. Specifically, when fluoroscopic imaging using the first X-ray imaging unit 30 is selected by the user, the image generation unit 51 generates an X-ray image (moving image) of an imaging region based on the detection signal output from the first X-ray detection unit 32. After the fluoroscopic imaging has been performed, the image generation unit 51 causes the display unit 60 to continuously display the LIH image 98.

The image generation unit 51 is also configured to cause the display unit 60 to display an irradiation field image 193 showing the range of the irradiation field. The irradiation field image 193 is an image showing a range 99 of the first irradiation field at a predetermined height position from the placement surface 10a of the tabletop 10. The irradiation field image 193 includes the LIH image 98 located in the range 99 of the first irradiation field.

The image generation unit 51 acquires the range 99 of the first irradiation field at the current time based on the irradiation field range information related to the range 99 of the first irradiation field. The image generation unit 51 also acquires a range 199 of the first irradiation field at the time of generation of the LIH image 98, based on the irradiation field range information at the time of generation of the LIH image 98. Then, the image generation unit 51 generates an irradiation field image 193 in which the LIH image 98 is placed at a position in the current range 99 of the first irradiation field corresponding to the position in the range 199 of the first irradiation field at the time of image generation. Note that the image generation unit 51 causes a portion 900 (see FIG. 13(b) and FIG. 15(b)) outside the range of the LIH image 98 in the irradiation field image 193 displayed on the display unit 60 to be displayed in black.

Note that, after the fluoroscopic imaging has been performed and before the tabletop 10 or the first X-ray imaging unit 30 is moved, the range 199 of the first irradiation field at the time of image generation and the current range 99 of the first irradiation field are the same. Therefore, after the fluoroscopic imaging has been performed and before the tabletop 10 or the first X-ray imaging unit 30 is moved, the LIH image 98 is displayed on the display unit 60 as the irradiation field image 193.

Furthermore, after the fluoroscopic imaging has been performed, when the tabletop 10 or the first X-ray imaging unit 30 is moved, the image generation unit 51 moves the position of the LIH image 98 in the irradiation field image 193 according to the moving direction and moving distance of the tabletop 10, or the moving direction and moving distance of the first X-ray imaging unit 30. That is, the image generation unit 51 moves the LIH image 98 in the irradiation field image 193 so as to follow the movement of the tabletop 10 or the first X-ray imaging unit 30, based on the current range 99 of the first irradiation field and the range 199 of the first irradiation field at the time of image generation. At this time, depending on the moving direction, moving distance of the tabletop 10 or the first X-ray imaging unit 30, and the dimension of the aperture of the collimator 31b, the moved LIH image 98 in the display area of the display unit 60 may not be displayed in its entirety, and only a part of it may be displayed in the display area.

Here, after the fluoroscopic imaging has been performed, when the tabletop 10 or the first X-ray imaging unit 30 is moved, the image generation unit 51 moves the position of the irradiation field image 193 showing the current range 99 of the first irradiation field according to the moving direction and moving distance of the tabletop 10, or the moving direction and moving distance of the first X-ray imaging unit 30. In other words, the image generation unit 51 is configured to move the irradiation field image 193 showing the current range 99 of the first irradiation field on the LIH image 98 on the display unit 60 in response to the movement of at least one of the first X-ray imaging unit 30 via the second support column drive unit 87 and the tabletop 10 via the first tabletop drive unit 84 and the second tabletop drive unit 85. Note that the second support column drive unit 87, the first tabletop drive unit 84, and the second tabletop drive unit 85 are an example of the “drive unit” in the claims.

Furthermore, as shown in FIG. 10 to FIG. 13, when fluoroscopic imaging using the first X-ray imaging unit 30 is selected by the user, after the fluoroscopic imaging has been performed, the image generation unit 51 is configured to cause the display unit 60 to display the interference information image 192 superimposed on the irradiation field image 193 including the LIH image 98. That is, the image generation unit 51 is configured to provide a notification that the tabletop unit-installed part 2 is interfering with the first irradiation field by causing the display unit 60 to display the irradiation field image 193 on which the interference information image 192 as the interference information 90 is superimposed.

Specifically, as shown in FIG. 13(b) and FIG. 15(b), the image generation unit 51 is configured to generate an interference information image 192 as the interference information 90 showing a range where the tabletop unit-installed part 2 interferes in the first irradiation field, based on the irradiation field range information related to the range of the first irradiation field and the member position information.

That is, as shown in FIG. 13(b), the image generation unit 51 generates the interference information image 192 as the interference information 90 showing the range where the tabletop moving rail 20 interferes in the first irradiation field, based on the irradiation field range information related to the range of the first irradiation field and the member position information related to the position of the tabletop moving rail 20. The interference information image 192 is, for example, an image with a visible color applied to the range where the tabletop moving rail 20 interferes in the first irradiation field. The display mode of the interference information image 192 is not particularly limited, and the interference information image 192 may be, for example, a frame image showing the outer edge of the range where the tabletop moving rail 20 interferes in the first irradiation field.

Furthermore, as shown in FIG. 15(b), the image generation unit 51 generates the interference information image 192 as the interference information 90 showing the range where the second X-ray detection unit moving member 21 interferes in the first irradiation field, based on the irradiation field range information related to the range of the first irradiation field and the member position information related to the position of the second X-ray detection unit moving member 21. The interference information image 192 is, for example, an image with a visible color applied to the range where the second X-ray detection unit moving member 21 interferes in the first irradiation field. The display mode of the interference information image 192 is not particularly limited, and the interference information image 192 may be, for example, a frame image showing the outer edge of the range where the second X-ray detection unit moving member 21 interferes in the first irradiation field.

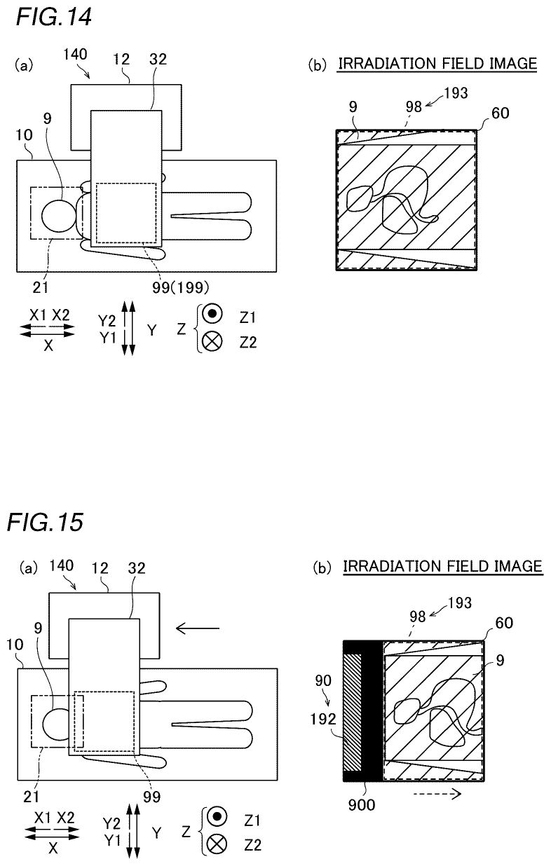

As shown in FIG. 12(b) and FIG. 14(b), the image generation unit 51 generates an irradiation field image 193 including the LIH image 98 and causes the display unit 60 to display the generated irradiation field image 193. Furthermore, in a state where the tabletop unit-installed part 2 interferes in the first irradiation field, as shown in FIG. 13(b) and FIG. 15(b), the image generation unit 51 generates an irradiation field image 193 including the LIH image 98, on which the interference information image 192 as the interference information 90 showing the range where the tabletop unit-installed part 2 interferes in the first irradiation field is aligned and superimposed, and causes the display unit 60 to display the generated irradiation field image 193.

### Control of Display of Interference Information Image by Control Unit

### Control of Display of Interference Information Image Related to Tabletop Moving Rail in Fluoroscopic Imaging Using First X-Ray Imaging Unit

With reference to FIG. 12 and FIG. 13, the control of the display of the interference information image 192 related to the tabletop moving rail 20 in fluoroscopic imaging using the first X-ray imaging unit 30 by the control unit 5 will be described.

The example shown in FIG. 12 and FIG. 13 is an example of the control of the display of the interference information image 192 related to the tabletop moving rail 20 when searching for a region of interest by moving the first X-ray imaging unit 30 in the Y1 direction after fluoroscopic imaging has been performed on the subject 9 in a supine position on the tabletop 10. Note that FIG. 12(a) and FIG. 13(a) are views of the tabletop 10 from the Z1 direction. In FIG. 12 and FIG. 13, no X-rays are irradiated from the first X-ray irradiation unit 31.

FIG. 12(a) is a schematic diagram showing the positional relationship among the subject 9, the first X-ray imaging unit 30, the tabletop 10, and the tabletop moving rail 20 before moving the first X-ray imaging unit 30 in the Y1 direction after fluoroscopic imaging has been performed. In FIG. 12(a), the tabletop moving rail 20, which is not visible to the user because it is disposed below the tabletop 10, is represented by a dash-dotted line. FIG. 12(b) is a schematic diagram of the irradiation field image 193 displayed on the display unit 60 before moving the first X-ray imaging unit 30 in the Y1 direction after fluoroscopic imaging has been performed. In this case, the irradiation field image 193 is the LIH image 98. In the states of FIG. 12(a) and FIG. 12(b), the tabletop moving rail 20 does not interfere with the first irradiation field.

In the state of FIG. 12(a), the image generation unit 51 acquires the range 99 of the first irradiation field at the current time based on the irradiation field range information related to the range of the first irradiation field. The image generation unit 51 also acquires the range 199 of the first irradiation field at the time of generation of the LIH image 98, based on the irradiation field range information at the time of generation of the LIH image 98. Then, the image generation unit 51 generates an irradiation field image 193 in which the LIH image 98 is placed at a position in the current range 99 of the first irradiation field corresponding to the position in the range 199 of the first irradiation field at the time of image generation. Then, as shown in FIG. 12(b), the image generation unit 51 causes the display unit 60 to display the irradiation field image 193 in which the LIH image 98 is placed.

FIG. 13(a) is a schematic diagram showing the positional relationship among the subject 9, the first X-ray imaging unit 30, the tabletop 10, and the tabletop moving rail 20 after moving the first X-ray imaging unit 30 in the Y1 direction. Also in FIG. 13(a), the tabletop moving rail 20, which is not visible to the user because it is disposed below the tabletop 10, is represented by a dash-dotted line. FIG. 13(b) is a schematic diagram of the irradiation field image 193 displayed on the display unit 60 after moving the first X-ray imaging unit 30 in the Y1 direction. As shown in FIG. 13(b), the irradiation field image 193 showing the current range 99 of the first irradiation field is moved on the LIH image 98 on the display unit 60 in response to the movement of the first X-ray imaging unit 30. In the states of FIG. 13(a) and FIG. 13(b), the tabletop moving rail 20 interferes with the first irradiation field.

In the state of FIG. 13(a), similarly to the state of FIG. 12(a), the image generation unit 51 generates an irradiation field image 193 in which the LIH image 98 is placed at a position in the current range 99 of the first irradiation field corresponding to the position in the range 199 of the first irradiation field at the time of image generation. Here, the image generation unit 51 generates the interference information image 192 because the tabletop moving rail 20 interferes in the first irradiation field, based on the irradiation field range information related to the range 99 of the first irradiation field and the member position information related to the position of the tabletop moving rail 20. The interference information image 192 is, as an example, an image with a visible color applied to the range where the tabletop moving rail 20 interferes in the first irradiation field. Then, as shown in FIG. 13(b), the image generation unit 51 causes the display unit 60 to display the irradiation field image 193 including the LIH image 98, on which the interference information image 192 is aligned and superimposed.

Note that in the state of FIG. 13(a), if the first X-ray imaging unit 30 is further moved in the Y1 direction, the irradiation field image 193, in which the interference information image 192 is superimposed on the LIH image 98, is displayed on the display unit 60.

### Control of Display of Interference Information Image Related to Second X-Ray Detection Unit Moving Member in Fluoroscopic Imaging Using First X-Ray Imaging Unit

With reference to FIG. 14 and FIG. 15, the control of the display of the interference information image 192 related to the second X-ray detection unit moving member 21 in fluoroscopic imaging using the first X-ray imaging unit 30 by the control unit 5 will be described.

The example shown in FIG. 14 and FIG. 15 is an example of the control of the display of the interference information image 192 related to the second X-ray detection unit moving member 21 when the first X-ray imaging unit 30 is moved in the X1 direction after fluoroscopic imaging has been performed on the subject 9 in a supine position on the tabletop 10. Note that FIG. 14(a) and FIG. 15(a) are views of the tabletop 10 from the Z1 direction. In FIG. 14 and FIG. 15, no X-rays are irradiated from the first X-ray irradiation unit 31.

FIG. 14(a) is a schematic diagram showing the positional relationship among the subject 9, the first X-ray imaging unit 30, the tabletop 10, and the second X-ray detection unit moving member 21 before moving the first X-ray imaging unit 30 in the X1 direction after fluoroscopic imaging has been performed. In FIG. 14(a), the second X-ray detection unit moving member 21, which is not visible to the user because it is disposed below the tabletop 10, is represented by a two-dot chain line. FIG. 14(b) is a schematic diagram of the irradiation field image 193 displayed on the display unit 60 before moving the first X-ray imaging unit 30 in the X1 direction after fluoroscopic imaging has been performed. In this case, the irradiation field image 193 is the LIH image 98. In the states of FIG. 14(a) and FIG. 14(b), the second X-ray detection unit moving member 21 does not interfere with the first irradiation field.

In the state of FIG. 14(a), the image generation unit 51 acquires the range 99 of the first irradiation field at the current time based on the irradiation field range information related to the range of the first irradiation field. The image generation unit 51 also acquires the range 199 of the first irradiation field at the time of generation of the LIH image 98, based on the irradiation field range information at the time of generation of the LIH image 98. Then, the image generation unit 51 generates an irradiation field image 193 in which the LIH image 98 is placed at a position in the current range 99 of the first irradiation field corresponding to the position in the range 199 of the first irradiation field at the time of image generation. Then, as shown in FIG. 14(b), the image generation unit 51 causes the display unit 60 to display the irradiation field image 193 in which the LIH image 98 is placed.

FIG. 15(a) is a schematic diagram showing the positional relationship among the subject 9, the first X-ray imaging unit 30, the tabletop 10, and the second X-ray detection unit moving member 21 after moving the first X-ray imaging unit 30 in the X1 direction. Also in FIG. 15(a), the second X-ray detection unit moving member 21, which is not visible to the user because it is disposed below the tabletop 10, is represented by a two-dot chain line. FIG. 15(b) is a schematic diagram of the irradiation field image 193 displayed on the display unit 60 after moving the first X-ray imaging unit 30 in the X1 direction. As shown in FIG. 15(b), the irradiation field image 193 showing the current range 99 of the first irradiation field is moved on the LIH image 98 on the display unit 60 in response to the movement of the first X-ray imaging unit 30. In the states of FIG. 15(a) and FIG. 15(b), the second X-ray detection unit moving member 21 interferes with the first irradiation field.

In the state of FIG. 15(a), similarly to the state of FIG. 14(a), the image generation unit 51 generates an irradiation field image 193 in which the LIH image 98 is placed at a position in the current range 99 of the first irradiation field corresponding to the position in the range 199 of the first irradiation field at the time of image generation. Here, the image generation unit 51 generates the interference information image 192 because the second X-ray detection unit moving member 21 interferes in the first irradiation field, based on the irradiation field range information related to the range 99 of the first irradiation field and the member position information related to the position of the second X-ray detection unit moving member 21. The interference information image 192 is, as an example, an image with a visible color applied to the range where the second X-ray detection unit moving member 21 interferes in the first irradiation field. Then, as shown in FIG. 15(b), the image generation unit 51 causes the display unit 60 to display the irradiation field image 193 including the LIH image 98, on which the interference information image 192 is aligned and superimposed.

Note that in the state of FIG. 15(a), if the first X-ray imaging unit 30 is further moved in the X1 direction, the irradiation field image 193, in which the interference information image 192 is superimposed on the LIH image 98, is displayed on the display unit 60.

In the present embodiment, when X-ray imaging using the first X-ray imaging unit 30 is selected by the user, after the X-ray imaging has been performed, the control unit 5 may be configured to cause the display unit 60 to display the interference information image 192 as the interference information 90 in the first irradiation field superimposed on an irradiation field image 193 including a final X-ray image. The final X-ray image means the last X-ray image generated in the X-ray imaging. When X-ray imaging using the first X-ray imaging unit 30 is selected by the user, after the X-ray imaging has been performed, the display unit 60 continuously displays the final X-ray image, which is the last generated X-ray image. In this case, the display unit 60 continuously displays the final X-ray image after the X-ray imaging has been performed. The final X-ray image is an example of the “X-ray image” in the claims.

Other configurations of the fourth embodiment are the same as those of the first embodiment.

### Effects of Fourth Embodiment

In the fourth embodiment, the following effects can be obtained.

In the fourth embodiment, as described above, the display unit 60 is configured to display the irradiation field image 193 that includes the X-ray image based on the detection signal detected by the X-ray detection unit and shows the range of the irradiation field, and the control unit 5 is configured to cause the display unit 60 to display the interference information image 192 as the interference information 90, which indicates the range where the tabletop unit-installed part 2 interferes in the first irradiation field, superimposed on the irradiation field image 193 including the LIH image 98. Thereby, for example, even when the tabletop unit-installed part 2 is interfering with the range of the X-ray irradiation field after the positioning of the X-ray irradiation field has been performed using the LIH image 98 displayed on the display unit 60, since the interference information image 192 indicating that the tabletop unit-installed part 2 is interfering with the first irradiation field is displayed on the display unit 60, the user can more easily recognize that the tabletop unit-installed part 2 is interfering with the first irradiation field.

Furthermore, in the fourth embodiment, as described above, the apparatus further includes a second support column drive unit 87, a first tabletop drive unit 84, and a second tabletop drive unit 85 that move at least one of the first X-ray imaging unit 30 and the tabletop 10, and the control unit 5 is configured to move the irradiation field image 193 showing the range 99 of the first irradiation field on the X-ray image on the display unit 60 in response to the movement of at least one of the first X-ray imaging unit 30 and the tabletop 10 by the second support column drive unit 87, the first tabletop drive unit 84, and the second tabletop drive unit 85. Thereby, since the irradiation field image 193 showing the range 99 of the first irradiation field is moved on the X-ray image on the display unit 60 so as to follow the movement of the first X-ray imaging unit 30 or the tabletop 10, positioning of the X-ray irradiation field can be easily performed after the irradiation of X-rays by the first X-ray imaging unit 30 is stopped, and an increase in radiation exposure dose during positioning of the X-ray irradiation field can be suppressed.

Other effects of the fourth embodiment are the same as those of the first embodiment.

### Modified Examples

It should be understood that the embodiments disclosed herein are illustrative and not restrictive in all respects. The scope of the present invention is indicated by the claims rather than by the description of the embodiments, and all modifications (modified examples) within the meaning and scope equivalent to the claims are intended to be included.

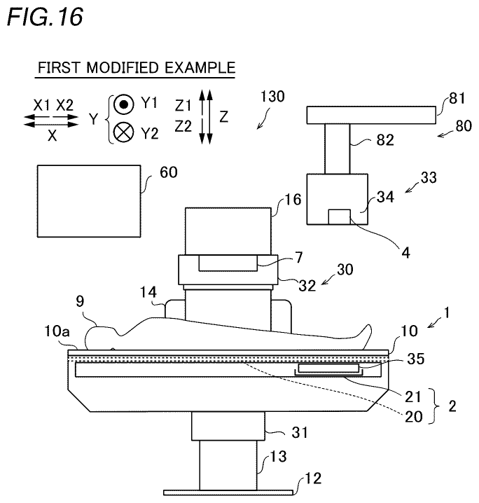

For example, in the first embodiment, an example was shown in which the optical imaging unit 4 is provided on the first X-ray detection unit 32 of the first X-ray imaging unit 30 and not provided on the second X-ray imaging unit 33, but the present invention is not limited to this. For example, as in an X-ray imaging apparatus 130 of a first modified example shown in FIG. 16 to FIG. 18, the optical imaging unit 4 (see FIG. 16) may be configured to be provided on the second X-ray irradiation unit 34 of the second X-ray imaging unit 33.

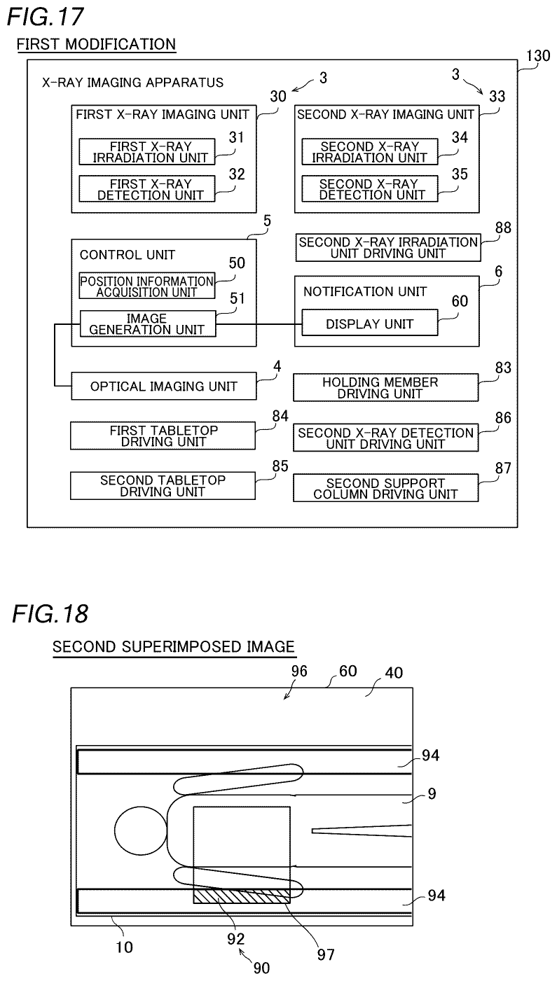

In the first modified example, as shown in FIG. 17, the control unit 5 is configured to cause the notification unit 6 to provide a notification that the tabletop moving rail 20 is interfering with a second irradiation field, based on irradiation field range information related to a range 97 of the second irradiation field of X-rays irradiated from the second X-ray irradiation unit 34 and member position information related to the position of the tabletop moving rail 20. Note that the range 97 of the second irradiation field includes the range of the X-ray irradiation field when X-rays are actually being irradiated from the second X-ray irradiation unit 34, and the range of the X-ray irradiation field when X-rays would be irradiated from the second X-ray irradiation unit 34.

When X-ray imaging or fluoroscopic imaging using the second X-ray imaging unit 33 is selected by the user, the position information acquisition unit 50 is configured to acquire the irradiation field range information related to the range 97 of the second irradiation field and the member position information related to the position of the tabletop moving rail 20.

Furthermore, when X-ray imaging or fluoroscopic imaging using the second X-ray imaging unit 33 is selected by the user, the image generation unit 51 is configured to generate a second superimposed image 96 (see FIG. 18) and to cause the display unit 60 of the notification unit 6 to display the generated second superimposed image 96. The second superimposed image 96 is an image in which an image 94 showing the position of the tabletop moving rail 20 as the tabletop unit-installed part 2, and an interference information image 92 as the interference information 90 showing a range where the tabletop moving rail 20 interferes in the second irradiation field are superimposed on an optical image 40 captured by the optical imaging unit 4.

When X-ray imaging or fluoroscopic imaging using the second X-ray imaging unit 33 is selected by the user, the image generation unit 51 acquires the optical image 40 captured by the optical imaging unit 4. Note that since the range 97 of the second irradiation field is represented in the optical image 40 by the visible light irradiated from the irradiation field lamp, the image generation unit 51 does not generate an image showing the range 97 of the second irradiation field. The image generation unit 51 generates the image 94 showing the position of the tabletop moving rail 20 based on the member position information related to the position of the tabletop moving rail 20. The image 94 showing the position of the tabletop moving rail 20 is, for example, a frame image showing the outer edge of the tabletop moving rail 20.

Furthermore, the image generation unit 51 is configured to generate an interference information image 92 as the interference information 90 showing the range where the tabletop moving rail 20 interferes in the second irradiation field, based on the irradiation field range information related to the range 97 of the second irradiation field and the member position information related to the position of the tabletop moving rail 20.

The image generation unit 51 generates a second superimposed image 96 in which the image 94 showing the position of the tabletop moving rail 20 is superimposed on the optical image 40, and causes the display unit 60 to display the generated second superimposed image 96. Furthermore, in a state where the tabletop unit-installed part 2 interferes in the second irradiation field, the image generation unit 51 generates a second superimposed image 96 in which the image 94 showing the position of the tabletop moving rail 20 and the interference information image 92 as the interference information 90 showing the range where the tabletop moving rail 20 interferes in the second irradiation field are superimposed on the optical image 40, and causes the display unit 60 to display the generated second superimposed image 96.

Other configurations of the first modified example are the same as those of the first embodiment.

In the first modified example, as described above, the X-ray irradiation unit includes the first X-ray irradiation unit 31 and the second X-ray irradiation unit 34, the X-ray detection unit includes the first X-ray detection unit 32 and the second X-ray detection unit 35, the X-ray imaging unit 3 includes the first X-ray imaging unit 30, which includes the first X-ray irradiation unit 31 disposed on the tabletop unit 1 and the first X-ray detection unit 32 disposed at a position facing the first X-ray irradiation unit 31 across the tabletop 10, and the second X-ray imaging unit 33, which includes the second X-ray detection unit 35 disposed on the tabletop unit 1 and the second X-ray irradiation unit 34 disposed on the side opposite to the second X-ray detection unit 35 across the tabletop 10, the tabletop unit-installed part 2 is the tabletop moving rail 20, and the control unit 5 is configured to cause the notification unit 6 to provide a notification that the tabletop moving rail 20 is interfering with the second irradiation field, based on the irradiation field range information related to the range 97 of the second irradiation field of X-rays irradiated from the second X-ray irradiation unit 34 and the member position information related to the position of the tabletop moving rail 20. Thereby, since the user can recognize that the tabletop moving rail 20 is interfering with the second irradiation field, it is possible to suppress an increase in radiation exposure dose due to retaking of X-ray images or extension of fluoroscopic imaging time caused by interference of the tabletop moving rail 20 with the second irradiation field.

Other effects of the first modified example are the same as those of the first embodiment.

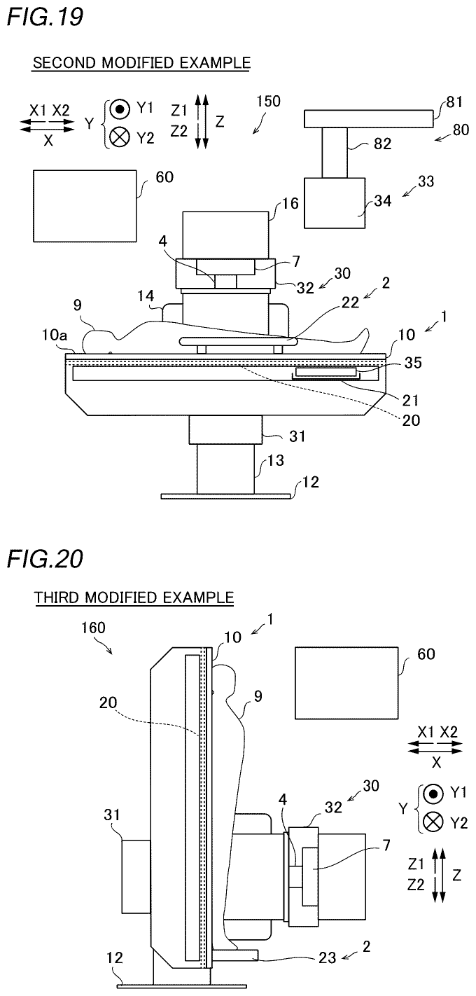

Furthermore, in the first embodiment, an example was shown in which the tabletop unit-installed part 2 includes the tabletop moving rail 20 and the second X-ray detection unit moving member 21, but the present invention is not limited to this. For example, as in an X-ray imaging apparatus 150 of a second modified example shown in FIG. 19, the tabletop unit-installed part 2 may be configured to include a gripping member 22.

In the second modified example, the gripping member 22 is provided on the tabletop unit 1 and is configured to be gripped by the subject 9. The gripping member 22 is configured to include a metal part with a lower X-ray transmittance than the portion of the tabletop unit 1 where the subject 9 is placed.

The position information acquisition unit 50 is configured to acquire, for example, member position information related to the position of the gripping member 22 based on the acquired position information of the tabletop 10. Then, when X-ray imaging or fluoroscopic imaging using the first X-ray imaging unit 30 is selected by the user, the image generation unit 51 is configured to generate a third superimposed image (not shown) and to cause the display unit 60 of the notification unit 6 to display the generated third superimposed image. The third superimposed image is an image in which an irradiation field image 93 showing the range 99 of the first irradiation field at a predetermined height position, an image showing the position of the gripping member 22, and an interference information image 92 as the interference information 90 showing a range where the gripping member 22 interferes in the first irradiation field are superimposed on an optical image 40 captured by the optical imaging unit 4.

Furthermore, as in an X-ray imaging apparatus 160 of a third modified example shown in FIG. 20, the tabletop unit-installed part 2 may be configured to include a support stand 23. In FIG. 20, for convenience of explanation, the illustration of the second X-ray imaging unit 33 is omitted.

The X-ray imaging apparatus 160 is configured to be capable of imaging the subject 9 in a supine state on the tabletop 10 according to the first embodiment shown in FIG. 1, and also capable of imaging the subject 9 in a standing state (upright position) as in the third modified example shown in FIG. 20. That is, in the X-ray imaging apparatus 160, the tabletop 10 and the first X-ray imaging unit 30 are configured to be rotatable around an axis that is orthogonal to the longitudinal direction of the tabletop 10 and oriented in the horizontal direction.

As shown in FIG. 20, when imaging the subject 9 in a standing state (upright position), the support stand 23 is provided on the tabletop unit 1 and is configured to support the subject 9. The support stand 23 is configured to include a metal part with a lower X-ray transmittance than the portion of the tabletop unit 1 where the subject 9 is placed.

The position information acquisition unit 50 is configured to acquire, for example, member position information related to the position of the support stand 23 based on the acquired position information of the tabletop 10. Then, when X-ray imaging or fluoroscopic imaging using the first X-ray imaging unit 30 is selected by the user, the image generation unit 51 is configured to generate a fourth superimposed image (not shown) and to cause the display unit 60 of the notification unit 6 to display the generated fourth superimposed image. The fourth superimposed image is an image in which an irradiation field image 93 showing the range 99 of the first irradiation field at a predetermined height position, an image showing the position of the support stand 23, and an interference information image 92 as the interference information 90 showing a range where the support stand 23 interferes in the first irradiation field are superimposed on an optical image 40 captured by the optical imaging unit 4.

Furthermore, in the first embodiment, an example was shown in which the control unit causes the display unit to display, as the interference information image, an image showing the interfering range with a semi-transparent color that allows the optical image to be visible, superimposed on the optical image, but the present invention is not limited to this. For example, the control unit may be configured to cause the display unit to display, as the interference information image, a frame image showing the outer edge of the interfering range superimposed on the optical image; or may be configured to cause the display unit to display, as the interference information image, an image showing the interfering range with an opaque color that does not allow the optical image to be visible, superimposed on the optical image; or may be configured to cause the display unit to display, as the interference information image, an image showing the interfering range with a blinking semi-transparent color that allows the optical image to be visible, superimposed on the optical image. The mode of the interference information image is not particularly limited.

Furthermore, the control unit may be configured to display an icon on the display unit indicating that the tabletop unit-installed part is interfering with the irradiation field, as the interference information image. That is, instead of superimposing an image showing the interfering range on the optical image, the control unit may be configured to display an icon such as a graphic on the display unit.

Furthermore, in the first embodiment, an example was shown in which the control unit causes the display unit to display the first superimposed image, in which an image showing the range of the first irradiation field, an image showing the position of the tabletop unit-installed part, and an interference information image as interference information showing the range where the tabletop unit-installed part interferes in the first irradiation field are aligned and superimposed on the optical image, but the present invention is not limited to this. For example, the control unit may be configured to display on the display unit a first superimposed image in which the interference information image is superimposed on the optical image, without superimposing the image showing the range of the first irradiation field and the image showing the position of the tabletop unit-installed part on the optical image, based on the irradiation field range information and the member position information. Also, for example, the control unit may be configured to display on the display unit a first superimposed image in which the image showing the range of the first irradiation field and the interference information image are superimposed on the optical image, without superimposing the image showing the position of the tabletop unit-installed part on the optical image, based on the irradiation field range information and the member position information.

Furthermore, in the first embodiment, an example was shown in which the control unit displays both the frame image showing the position of the tabletop moving rail and the frame image showing the position of the second X-ray detection unit moving member in the first superimposed image, but the present invention is not limited to this. For example, the control unit may be configured to display either the frame image showing the position of the tabletop moving rail or the frame image showing the position of the second X-ray detection unit moving member in the first superimposed image. In this case, the control unit may be configured to display either the interference information image for the tabletop moving rail or the interference information image for the second X-ray detection unit moving member in the first superimposed image.

### ASPECTS

Those skilled in the art will understand that the exemplary embodiments described above are specific examples of the following aspects.

### Item 1

An X-ray imaging apparatus, comprising:
an X-ray imaging unit including an X-ray irradiation unit that irradiates a subject with X-rays and an X-ray detection unit that detects X-rays transmitted through the subject;
a tabletop unit on which the subject is placed and which is movable relative to the X-ray imaging unit;
a tabletop unit-installed part that is provided on the tabletop unit and includes a material with a lower X-ray transmittance than a portion of the tabletop unit where the subject is placed;
a notification unit; and
a control unit that performs control to cause the notification unit to provide a notification that the tabletop unit-installed part is interfering with an irradiation field, based on irradiation field range information related to a range of an irradiation field of X-rays irradiated from the X-ray irradiation unit and member position information related to a position of the tabletop unit-installed part.

### Item 2

The X-ray imaging apparatus according to item 1, wherein the tabletop unit-installed part includes at least one of a tabletop moving rail that is provided at both ends in a transverse direction of the tabletop unit and slides a tabletop included in the tabletop unit in a longitudinal direction of the tabletop unit, an X-ray detection unit moving member that is provided on the tabletop unit, holds the X-ray detection unit placed on the tabletop unit, and moves the X-ray detection unit in the longitudinal direction of the tabletop unit, a gripping member that is provided on the tabletop unit and is gripped by the subject, and a support stand that is provided on the tabletop unit and supports the subject, and is configured to include a metal part with low X-ray transmittance.

### Item 3

The X-ray imaging apparatus according to item 1 or 2, wherein the notification unit includes a display unit, and
the control unit is configured to cause the display unit to display interference information indicating that the tabletop unit-installed part is interfering with the irradiation field, based on the irradiation field range information and the member position information.

### Item 4

The X-ray imaging apparatus according to item 3, further comprising an optical imaging unit that captures an optical image of the subject and the tabletop unit,
wherein the display unit is configured to display the optical image captured by the optical imaging unit, and
the control unit is configured to cause the display unit to display an interference information image as the interference information, which indicates a range where the tabletop unit-installed part interferes in the irradiation field, superimposed on the optical image.

### Item 5

The X-ray imaging apparatus according to any one of items 1 to 4, wherein the notification unit includes a speaker, and
the control unit is configured to cause the speaker to provide a notification that the tabletop unit-installed part is interfering with the irradiation field, based on the irradiation field range information and the member position information.

### Item 6

The X-ray imaging apparatus according to item 2, wherein the X-ray irradiation unit includes a first X-ray irradiation unit and a second X-ray irradiation unit,
the X-ray detection unit includes a first X-ray detection unit and a second X-ray detection unit,
the X-ray imaging unit includes a first X-ray imaging unit comprising the first X-ray irradiation unit disposed on the tabletop unit and the first X-ray detection unit disposed at a position facing the first X-ray irradiation unit across the tabletop, and a second X-ray imaging unit comprising the second X-ray detection unit disposed on the tabletop unit and the second X-ray irradiation unit disposed on a side opposite to the second X-ray detection unit across the tabletop,
the tabletop unit-installed part is the tabletop moving rail, and
the control unit is configured to cause the notification unit to provide a notification that the tabletop moving rail is interfering with the first irradiation field or the second irradiation field, based on the irradiation field range information related to a range of a first irradiation field of X-rays irradiated from the first X-ray irradiation unit or a range of a second irradiation field of X-rays irradiated from the second X-ray irradiation unit, and the member position information related to the position of the tabletop moving rail.

### Item 7

The X-ray imaging apparatus according to item 2 or 6, wherein the X-ray irradiation unit includes a first X-ray irradiation unit and a second X-ray irradiation unit,
the X-ray detection unit includes a first X-ray detection unit and a second X-ray detection unit,
the X-ray imaging unit includes a first X-ray imaging unit comprising the first X-ray irradiation unit disposed on the tabletop unit and the first X-ray detection unit disposed at a position facing the first X-ray irradiation unit across the tabletop, and a second X-ray imaging unit comprising the second X-ray detection unit disposed on the tabletop unit and the second X-ray irradiation unit disposed on a side opposite to the second X-ray detection unit across the tabletop,
the tabletop unit-installed part is a second X-ray detection unit moving member as the X-ray detection unit moving member, and
the control unit is configured to cause the notification unit to provide a notification that the second X-ray detection unit moving member is interfering with the first irradiation field, based on the irradiation field range information related to a range of a first irradiation field of X-rays irradiated from the first X-ray irradiation unit and the member position information related to the position of the second X-ray detection unit moving member.

### Item 8

The X-ray imaging apparatus according to any one of items 1 to 7, wherein the control unit is configured to cause the notification unit to provide a notification that the tabletop unit-installed part is interfering with the irradiation field, based on the irradiation field range information and the member position information, at least during X-ray imaging by the X-ray imaging unit.

### Item 9

The X-ray imaging apparatus according to item 3, wherein the display unit is configured to display an irradiation field image that includes an X-ray image based on a detection signal detected by the X-ray detection unit and shows a range of the irradiation field, and the control unit is configured to cause the display unit to display an interference information image as the interference information, which indicates a range where the tabletop unit-installed part interferes in the irradiation field, superimposed on the irradiation field image including the X-ray image.

### Item 10

The X-ray imaging apparatus according to claim 9, further comprising a drive unit that moves at least one of the X-ray imaging unit and a tabletop included in the tabletop unit, wherein the control unit is configured to move the irradiation field image showing the range of the irradiation field on the X-ray image on the display unit in response to movement of at least one of the X-ray imaging unit and the tabletop by the drive unit.

### DESCRIPTION OF REFERENCE NUMERALS

100, 110, 120, 130, 140, 150, 160 X-ray imaging apparatus
9 Subject
1 Tabletop unit
2 Tabletop unit-installed part
3 X-ray imaging unit
4 Optical imaging unit
5 Control unit
6 Notification unit
10 Tabletop
20 Tabletop moving rail
21 Second X-ray detection unit moving member
22 Gripping member
23 Support stand
30 First X-ray imaging unit
31 First X-ray irradiation unit
32 First X-ray detection unit
33 Second X-ray imaging unit
34 Second X-ray irradiation unit
35 Second X-ray detection unit
40 Optical image
60 Display unit
61 Speaker
84 First tabletop drive unit (drive unit)
85 Second tabletop drive unit (drive unit)
87 Second support column drive unit (drive unit)
90 Interference information
92, 192 Interference information image
98 LIH image (X-ray image)
193 Irradiation field image

1. An X-ray imaging apparatus, comprising:
an X-ray imaging unit including an X-ray irradiation unit that irradiates a subject with X-rays and an X-ray detection unit that detects X-rays transmitted through the subject;
a tabletop unit on which the subject is placed and which is movable relative to the X-ray imaging unit;
a tabletop unit-installed part that is provided on the tabletop unit and includes a material with a lower X-ray transmittance than a portion of the tabletop unit where the subject is placed;
a notification unit; and
a control unit that performs control to cause the notification unit to provide a notification that the tabletop unit-installed part is interfering with an irradiation field, based on irradiation field range information related to a range of an irradiation field of X-rays irradiated from the X-ray irradiation unit and member position information related to a position of the tabletop unit-installed part.

2. The X-ray imaging apparatus according to claim 1, wherein the tabletop unit-installed part includes at least one of a tabletop moving rail that is provided at both ends in a transverse direction of the tabletop unit and slides a tabletop included in the tabletop unit in a longitudinal direction of the tabletop unit, an X-ray detection unit moving member that is provided on the tabletop unit, holds the X-ray detection unit placed on the tabletop unit, and moves the X-ray detection unit in the longitudinal direction of the tabletop unit, a gripping member that is provided on the tabletop unit and is gripped by the subject, and a support stand that is provided on the tabletop unit and supports the subject, and is configured to include a metal part with low X-ray transmittance.

3. The X-ray imaging apparatus according to claim 1, wherein the notification unit includes a display unit, and
the control unit is configured to cause the display unit to display interference information indicating that the tabletop unit-installed part is interfering with the irradiation field, based on the irradiation field range information and the member position information.

4. The X-ray imaging apparatus according to claim 3, further comprising an optical imaging unit that captures an optical image of the subject and the tabletop unit,
wherein the display unit is configured to display the optical image captured by the optical imaging unit, and
the control unit is configured to cause the display unit to display an interference information image as the interference information, which indicates a range where the tabletop unit-installed part interferes in the irradiation field, superimposed on the optical image.

5. The X-ray imaging apparatus according to claim 1, wherein the notification unit includes a speaker, and
the control unit is configured to cause the speaker to provide a notification that the tabletop unit-installed part is interfering with the irradiation field, based on the irradiation field range information and the member position information.

6. The X-ray imaging apparatus according to claim 2, wherein the X-ray irradiation unit includes a first X-ray irradiation unit and a second X-ray irradiation unit,
the X-ray detection unit includes a first X-ray detection unit and a second X-ray detection unit,
the X-ray imaging unit includes a first X-ray imaging unit comprising the first X-ray irradiation unit disposed on the tabletop unit and the first X-ray detection unit disposed at a position facing the first X-ray irradiation unit across the tabletop, and a second X-ray imaging unit comprising the second X-ray detection unit disposed on the tabletop unit and the second X-ray irradiation unit disposed on a side opposite to the second X-ray detection unit across the tabletop,
the tabletop unit-installed part is the tabletop moving rail, and
the control unit is configured to cause the notification unit to provide a notification that the tabletop moving rail is interfering with the first irradiation field or the second irradiation field, based on the irradiation field range information related to a range of a first irradiation field of X-rays irradiated from the first X-ray irradiation unit or a range of a second irradiation field of X-rays irradiated from the second X-ray irradiation unit, and the member position information related to the position of the tabletop moving rail.

7. The X-ray imaging apparatus according to claim 2, wherein the X-ray irradiation unit includes a first X-ray irradiation unit and a second X-ray irradiation unit,
the X-ray detection unit includes a first X-ray detection unit and a second X-ray detection unit,
the X-ray imaging unit includes a first X-ray imaging unit comprising the first X-ray irradiation unit disposed on the tabletop unit and the first X-ray detection unit disposed at a position facing the first X-ray irradiation unit across the tabletop, and a second X-ray imaging unit comprising the second X-ray detection unit disposed on the tabletop unit and the second X-ray irradiation unit disposed on a side opposite to the second X-ray detection unit across the tabletop,
the tabletop unit-installed part is a second X-ray detection unit moving member as the X-ray detection unit moving member, and
the control unit is configured to cause the notification unit to provide a notification that the second X-ray detection unit moving member is interfering with the first irradiation field, based on the irradiation field range information related to a range of a first irradiation field of X-rays irradiated from the first X-ray irradiation unit and the member position information related to the position of the second X-ray detection unit moving member.

8. The X-ray imaging apparatus according to claim 1, wherein the control unit is configured to cause the notification unit to provide a notification that the tabletop unit-installed part is interfering with the irradiation field, based on the irradiation field range information and the member position information, at least during fluoroscopic imaging by the X-ray imaging unit.

9. The X-ray imaging apparatus according to claim 3, wherein the display unit is configured to display an irradiation field image that includes an X-ray image based on a detection signal detected by the X-ray detection unit and shows a range of the irradiation field, and the control unit is configured to cause the display unit to display an interference information image as the interference information, which indicates a range where the tabletop unit-installed part interferes in the irradiation field, superimposed on the irradiation field image including the X-ray image.

10. The X-ray imaging apparatus according to claim 9, further comprising a drive unit that moves at least one of the X-ray imaging unit and a tabletop included in the tabletop unit, wherein the control unit is configured to move the irradiation field image showing the range of the irradiation field on the X-ray image on the display unit in response to movement of at least one of the X-ray imaging unit and the tabletop by the drive unit.
